(19) United States
(12) Patent Application Publication
(10) Pub. No.: US 2008/0305186 A1
(43) Pub. Date: Dec. 11, 2008

(54) Method and Composition for the Treatment of Cardiac Hypertrophy

(75) Inventors: Harold R. Garner, Flower Mound, TX (US); Mounir Errami, Dallas, TX (US)

Correspondence Address: CHALKER FLORES, LLP, 2711 LBJ FRWY, Suite 1036, DALLAS, TX, 75234, US

(73) Assignee: BOARD OF REGENTS, THE UNIVERSITY OF TEXAS SYSTEM, Austin, TX (US)

(21) Appl. No.: 11/761,327

(22) Filed: Jun. 11, 2007

(51) Int. Cl. A61K 33/06; A61K 31/05; A61K 31/08; A61K 31/155; A61K 31/16; A61K 31/203; A61K 31/44; A61K 31/473; A61K 38/16; A61K 38/10; A61K 31/541; A61K 31/5415; A61K 31/55; A61K 31/59; A61K 31/65; A61K 31/661

(52) U.S. Cl. 424/682; 514/12; 514/122; 514/14; 514/152; 514/154; 514/167; 514/217; 514/226.2; 514/227.8; 514/279; 514/284; 514/616; 514/634; 514/722; 514/725; 514/734; 514/736

(57) ABSTRACT

The present invention includes compositions and methods treat a patient suffering from one or more symptoms of cardiac hypertrophy, hypertension and/or ischemia by administering a pharmaceutically effective amount of a pharmaceutical composition having an anti-epileptic drug and an antibiotic to the patient, for example, the anti-epileptic drug may be carbamazepine and the antibiotic may be doxycycline.

### STATEMENT OF FEDERALLY FUNDED RESEARCH

This invention was made with U.S. Government support under Contract No. 1HV028 1 85 awarded by the NIH/National Heart Lung and Blood Institute. The government has certain rights in this invention.

### TECHNICAL FIELD OF THE INVENTION

The present invention relates in general to the field of treatments for subjects presenting symptoms of cardiac risk, specifically, pharmaceutical compositions and methods of treatment for cardiac hypertrophy associated with myocardial infarction.

### BACKGROUND OF THE INVENTION

Without limiting the scope of the invention, its background is described in connection with treatments for cardiac hypertrophy associated with myocardial infarction, whether diagnosed as a separate component of myocardial infarction or even if not separately diagnosed. Cardiac hypertrophy includes the enlargement and damage of the heart often caused by the heart working harder to maintain the blood flow against an increased resistance. Although, the body can tolerate the increased blood pressure for some period of time, eventually, damage to the kidneys, the brain, the eyes can occur or death. Cardiac hypertrophy is a significant risk factor for the development of congestive heart failure (CHF).

The repercussions of hypertension are diverse. If untreated, hypertension leads to an increased workload on the heart, and often results in a variety of cardiovascular disorders, e.g., angina pectoris, cardiac hypertrophy, coronary vascular diseases, ischemic heart injury, and, in more severe cases, myocardial infarction, heart failure and death.

Medication therapy is often used to treated hypertion and includes a number of oral and parenteral medications. For example, Beta-Blockers (beta-adrenergic blockers) are commonly used to reduce the sympathetic nerve input to the heart to cause the heart to beat less often per minute and with less force. Alpha-blockers (alpha-adrenergic blockers) target the nervous system to relax blood vessels, allowing blood to pass more easily. Diuretics are used to lower systemic blood pressure by reducing the plasma volume by causing the body to excrete water and salt. Angiotensin Converting Enzyme (ACE) lowers blood pressure by inhibiting the production of angiotensin II that normally causes blood vessels to narrow. Calcium channel blockers keep calcium from entering the muscle cells of the heart and blood vessels and vasodilators are used to relax the muscle in the blood vessel walls and lower blood pressure. Medication therapy can involve the treatment with a single agent (e.g., monotherapy) or in combination with other agents. However, most of these agents ameliorate the symptoms but not curing the diseases.

### SUMMARY OF THE INVENTION

The present inventors recognized that anticonvulsants or anti-epileptic drugs may be used to attenuated cardiac hypertrophy; and the combination of anti-epileptic drugs (e.g., carbamazepine) and antibiotics (e.g., doxycycline) further arrogated the hypertrophic phenotype and survival increased. Carbamazepine mediates these beneficial effects by interfering with β-adrenergic signaling. The combination of doxycycline and carbamazepine operate by differing modes of action upon both the β-adrenergic and α-adrenergic pathways to contribute to the observed synergy.

The present invention provides methods and compositions for the treatment of cardiac hypertrophy (hereafter referred to as CH). β-blockers have been used as a therapy to attenuate cardiac hypertrophy due in part to the involvement of β-adrenergic signaling in the development of cardiac hypertrophy. A down stream effector (adenylate cyclase) of the β-adrenergic pathway, also plays a role in the development of cardiac hypertrophy. Carbamazepine has been shown to abrogate both basal and forskolin-stimulated cAMP production by inhibiting adenylate cyclase and its downstream effects.

The present invention provides a method and composition for the treatment of cardiac hypertrophy using an anticonvulsant (e.g., the anti-epileptic drug carbamazepine) to modulate the development of cardiac hypertrophy. The present invention also provides a method of attenuating hypertrophy by providing carbamazepine in combination with the antibiotic doxycycline. The present invention may be used to treat cardiac hypertrophy resulting from myocardial infarction, whether diagnosed as a separate component of myocardial infarction or even if not separately diagnosed.

Prior to the discovery by the present inventors and their development of the novel compositions and methods of treatment, an anticonvulsant alone or in combination with an antibiotic has never been used to treat cardiovascular disease and/or hypertension nor have they ever given any indication that they could be used to or would have any affect on cardiovascular disease or hypertension

Carbamazepine is in a class of medications called anticonvulsants or anti-epileptic drug and it works by reducing abnormal excitement in the brain. Generally, carbamazepine has been used as an anticonvulsant primarily in the treatment of epilepsy and as a mood-stabilizing drug for the treatment of bipolar disorder. Carbamazepine are also used to treat episodes of mania, frenzied, abnormally excited, irritated moods, and mixed episodes when mania and depression are experienced at the same time in patients with bipolar I disorder. In addition, carbamazepine has been used to treat schizophrenia and trigeminal neuralgia (a condition that causes facial nerve pain). The mechanism of action of carbamazepine is relatively well understood and involves the stabilization of sodium channels to reduce the available open able sodium channels.

U.S. Pat. No. 6,977,253, entitled, “Methods for the treatment of bipolar disorder using carbamazepine” teaches carbamazepine, in extended release form, that is useful in the treatment of patients suffering from bipolar disorder. In order to minimize the time it takes to reach efficacy, carbamazepine, in extended release form, can be administered to the patient at an initial daily dose, which is then increased in daily increments until clinical efficacy is achieved.

U.S. Pat. No. 6,572,889, entitled, “Controlled release solid dosage carbamazepine formulations” includes a polymer or copolymer composition derived from one or more unsaturated carboxylic acids that is cross-linked and carbamazepine in conjunction with conventional materials such as fillers, excipients and surface active agents is disclosed. Solid dosage forms of immediate and sustained release tablets containing the polymer or copolymer compositions can be formed by wet granulation or wet granulation followed by blending with direct compression ingredients. The polymer or copolymer, as a controlled release agent, can enhance controlled-release properties while meeting acceptable release rates as specified by the USP. There is no indication that any of these compositions are effective in treating cardiovascular disease and hypertension.

Another compound, doxycycline, is a member of the tetracycline antibiotics family and is commonly used to treat various infections, e.g., pneumonia, respiratory tract infections, Lyme disease, acne; infections of skin, genital, urinary tract infections, gonorrhea, inflammatory diseases, chlamydia, periodontitis, and others. It is also used to prevent malaria and works by preventing the growth and spread of bacteria. The mechanism of action of doxycycline is relatively well understood and involves the modulation of protein synthesis.

For example, U.S. Pat. No. 7,112,578, entitled, “Methods and compositions for treatment of inflammatory disease” teaches compositions useful for treating inflammatory diseases, local inflammation and dermal irritation and include cetyl myristoleate compounds or related compounds and at least one compound useful for treatment of inflammatory disease, such as tetracycline compounds, Cox-2 inhibitors, non-steroidal anti-inflammatory drugs (NSAIDs), corticosteroids, local anaesthetics, chelating agents, matrix metalloprotease inhibitors, inhibitors of inflammatory cytokines, glucosamine, chondroitin sulfate and collagen hydrolysate.

U.S. Pat. No. 7,008,631, entitled, “Methods of simultaneously treating ocular rosacea and acne rosacea” teaches a method for simultaneously treating ocular rosacea and acne rosacea in a human in need thereof comprising administering systemically to said human a tetracycline compound in an amount that is effective to treat ocular rosacea and acne rosacea but has substantially no antibiotic activity. Again, there is no indication that any of these compositions are effective in treating cardiovascular disease and hypertension.

The present inventors recognized that carbamazepine was given in combination with the antibiotic doxycycline, which inhibits matrix metalloproteinases (MMPs), a better therapeutic outcome was observed (based on normalized heart-to-body weight and heart-to-tibia length ratios) than for either drug alone. Additionally, the combination therapy resulted in a three-fold increase in the survival rate. In support of a role for carbamazepine as a β-adrenergic antagonist, a lower heart rate was observed in mice treated with carbamazepine alone or in combination with doxycycline. This effect was not observed for mice treated with doxycycline alone to indicate that carbamazepine specifically attenuated the positive chronotropic effects of isoproterenol, a drug administered to mice to induce hypertrophy. Likewise, ISO-induced CREB activation was inhibited by carbamazepine alone and the drug combination, but not by doxycycline alone. Doxycycline, however apparently contributed to inhibition of the β-adrenergic signaling pathway. Furthermore, doxycycline also up-regulated the Adra1b, an α-adrenergic receptor, that is known to be beneficial to the heart.

However, until the discovery by the present inventors there has been no indication that carbamazepine alone or in combination with doxycycline (or any anticonvulsant alone or in combination with an antibiotic) could be used to treat cardiovascular disease or hypertension or that such a combination would have any affect on cardiovascular disease or hypertension.

The present invention provides a pharmaceutical composition having carbamazepine and doxycycline. The pharmaceutical composition may include pharmaceutically effective amounts of each compound. Another example includes a single dose pharmaceutical composition (e.g., tablet, caplet, capsule, mini tab, as well as other pharmaceutical compositions known to the skilled artisan in single or multidose forms) that includes a pharmaceutically effective amounts of carbamazepine and doxycycline.

The present invention also provides a pharmaceutical composition to ameliorate one or more symptoms of cardiac hypertrophy and includes an anti-epileptic drug and an antibiotic. A method of treating patient with hypertension and/or ischemia is also provided by the present invention. The method includes administering a pharmaceutically effective amount of a pharmaceutical composition having an anti-epileptic drug and an antibiotic, for example, the anti-epileptic drug may be carbamazepine and the antibiotic may be doxycycline.

The present invention includes a method for treating a patient suffering from cardiac hypertrophy by administering to the patient a pharmaceutically effective amount of an anti-epileptic drug or a pharmaceutically acceptable salt thereof and a pharmaceutically effective amount of an antibiotic or a pharmaceutically acceptable salt thereof. Another example includes a method for attenuating one or more complications of hypertension by administering a pharmaceutically effective amount of a first compound to affect a β-adrenergic pathway and administering a pharmaceutically effective amount of a second compound to affect a α-adrenergic pathway.

### BRIEF DESCRIPTION OF THE DRAWINGS

For a more complete understanding of the features and advantages of the present invention, reference is now made to the detailed description of the invention along with the accompanying figures and in which:

FIG. 1 is a graph of the comparative effects of doxycycline and carbamazepine on the heart to tibia length ratio;

FIGS. 2A, 2B and 2C are images of histological cross sections of mice hearts of carbamazepine treated and untreated mice;

FIG. 3 is a graph of the added therapeutic benefits of the combination of doxycycline and carbamazepine on cardiac hypertrophy;

FIG. 4A is a graph of the heart size reading after death and FIG. 4B is a Kaplan survival curve; and

FIG. 5 is a graph of the heart rate variation over course of treatment with isoproterenol; doxycycline and isoproterenol; carbamazepine and isoproterenol; or isoproterenol and doxycycline and carbamazepine.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present inventors recognized a need for a method and composition to treat condition that can follow myocardial infarctions, including cardiac hypertrophy. The heart can respond by increasing the load on a portion of the heart to compensate for the area damaged because of the infarction. The present invention provides an effective treatment for cardiac hypertrophy whether associated with myocardial infarction or diagnosed separately.

The present invention includes pharmaceutically compositions and methods of treatment by administering a pharmaceutically effective amount of an anti-epileptic drug (or a pharmaceutically acceptable salt thereof) alone or in combination with a pharmaceutically effective amount of an antibiotic (or a pharmaceutically acceptable salt thereof).

The anti-epileptic drug or anti-seizure agents may be used alone or in combination and include carbamazepine, oxcarbazepine, valproic acid and modifications or substitutions thereof. Other anti-seizure agents that may also be used in this fashion include: phenytoin, acetazolamide, chloropromazine hydrochloride, clonazepam, diazepam, dilantin, dimenhydrinate, diphenhydramine hydrochloride, ephedrine sulfate, divalproex sodium, ethosuximide, ethotoin BP, felbamate, magnesium sulfate, mephenyloin, mephobarbital, paramethadione, phenobarbital sodium, phenyloin sodium, primidone, sodium bromide, trimethadione, substituted dibenzoxazepines and valproate sodium. Similarly, the antibiotic may be used alone or in combination and includes doxycycline, minocycline, tetracycline, and modifications or substitutions thereof. The skilled artisan will recognize that other antibiotics may also be used.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

The present invention provides a pharmaceutical composition having an anti-epileptic drug and an antibiotic to ameliorate one or more symptoms of cardiac hypertrophy. The anti-epileptic drug includes carbamazepine and the antibiotic includes doxycycline. The anti-epileptic drug and the antibiotic can be administered together in a single pharmaceutical composition, separate single pharmaceutical composition in a multi layered composition, a bilayered composition, a mixture of compositions, a polymer matrix, a particle or nanoparticle having a mixture of anti-epileptic drugs and antibiotics thereon, a mixture of particles, polymer matrixes or nanoparticles each having an anti-epileptic drug and/or an antibiotic.

The compositions of the present invention exist in a suitable form for delivery, e.g., as a pharmaceutically acceptable salt of an organic or inorganic acid, e.g., hydrochloride, sulfate, hemi-sulfate, phosphate, nitrate, acetate, oxalate, citrate, maleate, mesylate, etc. Also, where an appropriate acidic group is present on a compound of the invention, a pharmaceutically acceptable salt of an organic or inorganic base can be employed such as an ammonium salt, or salt of an organic amine, or a salt of an alkali metal or alkaline earth metal such as a potassium, calcium or sodium salt.

The compositions of the present invention may be formulated into any of many possible dosage forms such as, but not limited to, tablets, capsules, gel capsules, liquid syrups and soft gels. The compositions of the present invention may be formulated as suspensions in aqueous, non-aqueous or mixed media. Aqueous suspensions may further contain substances that increase the viscosity of the suspension including, e.g., sodium carboxymethylcellulose, sorbitol and/or dextran. The suspension may optionally contain stabilizers. Furthermore, the percentage of therapeutic compounds in the compositions and preparations may, of course, be varied as will be known to the skilled artisan. The amount of the therapeutic compound in such therapeutically useful compositions is such that a suitable dosage will be obtained.

Other additives may include conventional additives used in pharmaceutical compositions, and are well known in the art. Such additives include, e.g.: anti-adherents (anti-sticking agents, glidants, flow promoters, lubricants) such as talc, magnesium stearate, fumed silica), micronized silica, polyethylene glycols, surfactants, waxes, stearic acid, stearic acid salts, stearic acid derivatives, starch, hydrogenated vegetable oils, sodium benzoate, sodium acetate, leucine, PEG-4000 and magnesium lauryl sulfate.

In some formulations, the additives may include chelating agents (e.g., EDTA and EDTA salts); colorants or opaquants (e.g., titanium dioxide, food dyes, lakes, natural vegetable colorants, iron oxides, silicates, sulfates, magnesium hydroxide and aluminum hydroxide); coolants (e.g., trichloroethane, trichloroethylene, dichloromethane, fluorotrichloromethane); cryoprotectants (e.g., trehelose, phosphates, citric acid, tartaric acid, gelatin, dextran and mannitol); and diluents or fillers (e.g., lactose, mannitol, talc, magnesium stearate, sodium chloride, potassium chloride, citric acid, spray-dried lactose, hydrolyzed starches, directly compressible starch, microcrystalline cellulose, cellulosics, sorbitol, sucrose, sucrose-based materials, calcium sulfate, dibasic calcium phosphate and dextrose). Yet other additives may include disintegrants or super disintegrants; hydrogen bonding agents, such as magnesium oxide; flavorants or desensitizers.

Suitable excipients are those used commonly to facilitate the processes involving the preparation of the solid carrier, the encapsulation coating or the pharmaceutical dosage form. These processes include agglomeration, air suspension chilling, air suspension drying, balling, coacervation, comminution, compression, pelletization, cryopelletization, extrusion, granulation, homogenization, inclusion complexation, lyophilization, nanoencapsulation, melting, mixing, molding, pan coating, solvent dehydration, sonication, spheronization, spray chilling, spray congealing, spray drying, or other processes known in the art. The excipients may also be pre-coated or encapsulated, and are well known in the art.

The carrier of the present invention may be a powder or a multiparticulate, such as a granule, a pellet, a bead, a spherule, a beadlet, a microcapsule, a millisphere, a nanocapsule, a nanosphere, a microsphere, a platelet, a minitablet, a tablet or a capsule. A carrier may be a finely divided (e.g., milled, micronized, nanosized, precipitated) form of a matrix on which the active ingredient is disposed. Such matrix may be formed of various materials known in the art, such as, sugars, e.g., lactose, sucrose or dextrose; polysaccharides, e.g., maltodextrin or dextrates; starches; cellulosics, e.g., microcrystalline cellulose or microcrystalline cellulose/sodium carboxymethyl cellulose; inorganics, e.g., dicalcium phosphate, hydroxyapitite, tricalcium phosphate, talc, or titania; and polyols, e.g., mannitol, xylitol, sorbitol or cyclodextrin. It should be emphasized that a substrate need not be a solid material, although often it will be a solid.

The composition of the present invention can be coated with one or more enteric coatings, seal coatings, film coatings, barrier coatings, compress coatings, fast disintegrating coatings, or enzyme degradable coatings. Multiple coatings may be applied for desired performance. Further, some actives may be provided for slow release, pulsatile release, controlled release, extended release, delayed release, targeted release, synchronized release, or targeted delayed release. For release/absorption control, solid carriers can be made of various component types and levels or thicknesses of coats, with or without an active ingredient. Such diverse solid carriers can be blended in a dosage form to achieve a desired performance.

Control of the release of drugs from drug-resin complexes has been achieved by the direct application of a diffusion barrier coating to particles of such complexes, provided that the drug content of the complexes was above a critical value. Any coating procedure that provides a contiguous coating on each particle of drug-resin complex without significant agglomeration of particles may be used. Measurements of particle size distribution before and after coating showed that agglomeration of particles was insignificant. Dosage forms of the compositions of the present invention can also be formulated as enteric coated delayed release oral dosage forms, i.e., as an oral dosage form of a pharmaceutical composition as described herein that uses an enteric coating to affect release in the lower gastrointestinal tract. The enteric coated dosage form may be a compressed or molded or extruded tablet/mold (coated or uncoated) containing granules, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

The coating may also contain a plasticizer and possibly other coating excipients such as colorants, talc, and/or magnesium stearate, which are well known in the art. Suitable plasticizers include: triethyl citrate (citroflex 2), triacetin (glyceryl triacetate), acetyl triethyl citrate (citroflec A2), carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the lower intestinal tract is reached.

Colorants, detackifiers, excipients, surfactants, antifoaming agents, lubricants, stabilizers such as hydroxy propyl cellulose or methylated cellulose, acid/base may be added to the coatings besides plasticizers to solubilize or disperse the coating material, and to improve coating performance and the coated product.

The solid pharmaceutical compositions of the present invention may include optionally one or more excipients, sometimes referred to as additives. The excipients may be contained in an encapsulation coat, or can be part of the solid carrier, such as coated to an encapsulation coat, or contained within the components forming the solid carrier. Alternatively, the excipients can be contained in the pharmaceutical composition but not part of the solid carrier itself. For example, the composition of the present invention may be made by a pelletization process, which typically involves preparing a molten solution of the composition of the solid carrier or a dispersion of the composition of the solid carrier solubilized or suspended in an aqueous medium, an organic solvent, a supercritical fluid, or a mixture thereof.

Cardiac Hypertrophy develops in response to biomechanical stress, such as prolonged arterial pressure overload or valvular heart disease, and is characterized by contractile dysfunction, decreased heart performance, and a significantly higher risk for heart failure, ischemic heart disease, and sudden death (1)(2). A reduction in the mass of the left ventricle greatly improves prognosis, independent of treatment type (3)(4) and is thus accepted as standard metric to assess the efficacy of therapy. The process of cardiac hypertrophy development is complicated, with multiple different signaling pathways capable of conducting stress stimuli to promote the hypertrophic response (5)(6)(7)(8)(9). Perhaps the best characterized of these signals is β-adrenergic stimulation, a major hypertrophic stimulus mediated via a G protein-coupled receptor that activates adenylate cyclase and subsequently cAMP production.

Isoproterenol (ISO), a β-adrenergic agonist that induces cardiac hypertrophy in mice, has been previously shown to increase cAMP production in cultured myocytes, comparable to forskolin-induced cAMP levels (10). Similarly, disruption of the gene encoding adenyalet cyclase has been shown to prevent isoproterenol—or pressure overload-induced cardiac hypertrophy (11). β-blockers are well-established as therapies that counter the consequences of hypertension and hypertrophy by preventing stimulation of this pathway and subsequently improving the survival rates of patients suffering from hypertrophy or heart failure (12)(13)(14). Further, this strategy might re-establish a favorable genetic expression pattern, such as causing up-regulation of previously depressed genes that encode potentially beneficial proteins. β-blockers for instance have been shown to cause up-regulation of α-myosin heavy chain (α-MHC) and the Ca²⁺ transporter SERCA2a, which are involved in cardiomyocyte contraction and relaxation (15)(16).

In order to identify new therapeutic targets for cardiac hypertrophy, a computational program IRIDESCENT was used to detect previously unknown relationships between medical objects (e.g., small molecules, phenotypes, and genes) in PubMed (17). This novel method of data mining was been shown to be a useful tool for identifying potential drug candidates, e.g., it previously predicted the known relationship between chlorpromazine and cardiac hypertrophy (18). Several therapeutic candidates were suggested, based on their published modes of action and potential for targeting pathways known to be important for cardiac hypertrophy. These included the antibiotic doxycycline (DOX), which inhibits MMPs, and the anti-epileptic carbamazepine (CBZ).

Another example of the present invention includes a method for attenuating one or more complications of hypertension by administering a pharmaceutically effective amount of a first compound to inhibit a matrix metalloproteases. A matrix metalloproteinases inhibiter or matrix metalloproteases (MMPs) inhibiter (e.g., doxycycline) are known to be involved in fibrosis and tissue remodeling. Generally, MMPs are zinc-dependent endopeptidases and include adamalysins, serralysins and astacins and belong to a larger family of proteases known as the metzincin superfamily. The present invention includes doxycycline which is a matrix metalloproteinases inhibiter; however, other matrix metalloproteinases inhibiter may be used in the present invention (e.g., prinomastat (AG3340; Agouron/Pfizer), BAY 12-9566 (Bayer Corp.), batimistat (BB-94; British Biotech, Ltd,), BMS-275291 (formerly D2163; Celltech/Bristol-Myers Squibb), marimastat (BB 2516; British Biotech, Ltd./Schering-Plough), MMI270(B) (formerly CGS-27023A; Novartis), and Metastat (COL-3; CollaGenex)). In addition, metzincin superfamily inhibitors may also be used in the present invention. Therefore, other matrix metalloproteinases inhibiters or combinations of inhibitors may be used in the present invention to affect matrix metalloproteinases activity.

Carbamazepine has been shown to abrogate both basal and forskolin-stimulated cAMP production by inhibiting adenylate cyclase and its downstream effects (19). The present inventors recognized that the use of both drugs in a mouse model of cardiac hypertrophy significantly attenuated hypertrophy. The present inventors recognized that the use of both carbamazepine and doxycycline administered in combination, the hypertrophic phenotype was further arrogated and survival increased. Carbamazepine mediates these beneficial effects by interfering with β-adrenergic signaling and differing modes of action upon both the β- and α-adrenergic pathways by carbamazepine and doxycycline contributed to the observed synergy of the two drugs.

All animal and mouse studies and/or models of cardiac hypertrophy were conducted in accordance with the standards set forth in the Guide for the Care and Use of Laboratory Animals (NIH Publication No. 85-23, revised 1996) and were approved by our Institutional Animal Care and Use Committee. Eight week-old C57BL/6 male mice (Jackson Laboratory) were given isoproterenol (Sigma Aldrich) at 40 mg·kg⁻¹d⁻¹ administered S.Q. via micro-osmotic pump insertion (ALZET 1007D). Briefly, animals were anesthetized with isoflurane (1.5%) and oxygen (98.5%) using an animal ventilator (Surgivet), an incision (1 cm) was made on the back between the shoulder blades, and micro-osmotic pumps containing isoproterenol dissolved in a saline solution (0.9% NaCl) were inserted into the infrascapular subcutaneous tissue.

Administration of doxycycline and carbamazepine. Doxycycline was given in drinking water at 6 mg mL⁻¹ (Sigma Aldrich) containing 5% sucrose unless specified otherwise. Control animals were given 5% sucrose water. Carbamazepine was given in rodent chow at 0.25% unless specified otherwise. Briefly, chow was crunched in powder and then mixed with carbamazepine. Water was added to the mix 0.8:1 (water weight to powder weight ratio) and the resulting paste diced and heated at 60° C. overnight.

Microarray Sample Preparation and Analysis. Animal hearts were rapidly removed, and the atria and right ventricles were cut and immediately plunged into TRIzol Reagent (Life Technologies). Total RNA was isolated following the manufacturer's instructions, purified by phenol-chloroform extraction and then ethanol precipitation, and 20 μg further processed for microarray analysis. Briefly, cDNA synthesis, in vitro transcription, and labeling and fragmentation to produce the oligonucleotide probes were performed as instructed by the GeneChip manufacturer (Affymetrix). The probes were first hybridized to a test array (Affymetrix) and then to the GeneChip Mouse Genome 430 2.0 Array, using the GeneChip Hybridization Oven 640. The chips were washed in a GeneChip Fluidics Station 450 (Affymetrix), and the results were visualized with a GeneChip G7 scanner (Affymetrix). RMA normalization, pairwise comparisons, Student's t test and Benjamini and Hochberg correction were subsequently performed using GeneSifter (VizX Labs, Seattle, Wash.) and Spotfire DecisionSite 8.3 (Spotfire, Inc., Somerville, Mass.).

Real-time reverse transcriptase-polymerase chain reaction (RT-PCR). Real-time quantitative RT-PCR was performed in the iCycler iQ multi-Color real-time PCR detection system (Bio-Rad, Hercules, Calif.) using SYBR Green I dye (Qiagen, Valencia, Calif.) as described by the manufacturer. Briefly, 100 ng of RNA was placed into a 25 μl reaction volume containing 2.5 μl of each primer set (Quantitect Primer Assays, Qiagen), 12.5 μl SYBER Green PCR master mix, and 0.25 μl reverse transcriptase. A typical protocol included reverse transcription at 50° C. for 30 minutes and a denaturation step at 95° C. for 15 minutes followed by 35 cycles with 94° C. denaturation for 15 seconds, 55 C annealing for 30 seconds and 72 C extension for 30 seconds. Detection of the fluorescent product was performed at the end of the extension period at 60° C. for 20 seconds. To confirm amplification specificity, the PCR products were subjected to a melting curve analysis. Negative controls containing water instead of RNA were concomitantly run to confirm that the samples were not cross-contaminated. Targets were normalized to reactions performed using Quantitect GAPDH primer assay (Qiagen), and fold change was determined using the comparative threshold method (20).

Histology. Animal hearts were excised, fixated with 10% phosphate-buffered formalin for 48 hours, and then embedded in paraffin. Cross-sectional slices in the minor axis were obtained with a microtome and the slices stained using Mayer's hematoxylin and eosin (H&E).

Western Blots. The antibodies for Adra1b and GAPDH were purchased from Santa Cruz Biochemical Co. (Santa Cruz, Calif.). All other antibodies were purchased from Cell Signaling Technology, and Western blot analysis was performed as previously described (21). Briefly, equal amounts of total protein were loaded and separated on sodium dodecyl sulfate (SDS)—10% polyacrylamide gels and then transferred to nitrocellulose membranes. Membranes were blocked with 5% milk and washed in 1× Tween (0.1%)—Tris-buffered saline (TTBS) three times for 5 minutes each. Primary antibodies diluted 1:1000 in 5% milk or bovine serum albumin (BSA) (prepared in 1×TTBS) were allowed to incubate overnight at 4° C. After washing, horseradish peroxidase (HRP)-conjugated secondary antibody (Cell Signaling Technology) was diluted 1:2000 in 5% milk and applied to membranes. Subsequently, membranes were washed and a chemiluminescence substrate (Pierce, Rockford Ill.) was applied and allowed to incubate at room temperature for 5 minutes.

Statistical analysis of the data includes values presented are expressed in mean ±S.E.M. All comparisons between groups were performed using a one way ANOVA followed by the Newman-Keuls test. Differences were considered to be statistically significant when P<0.05.

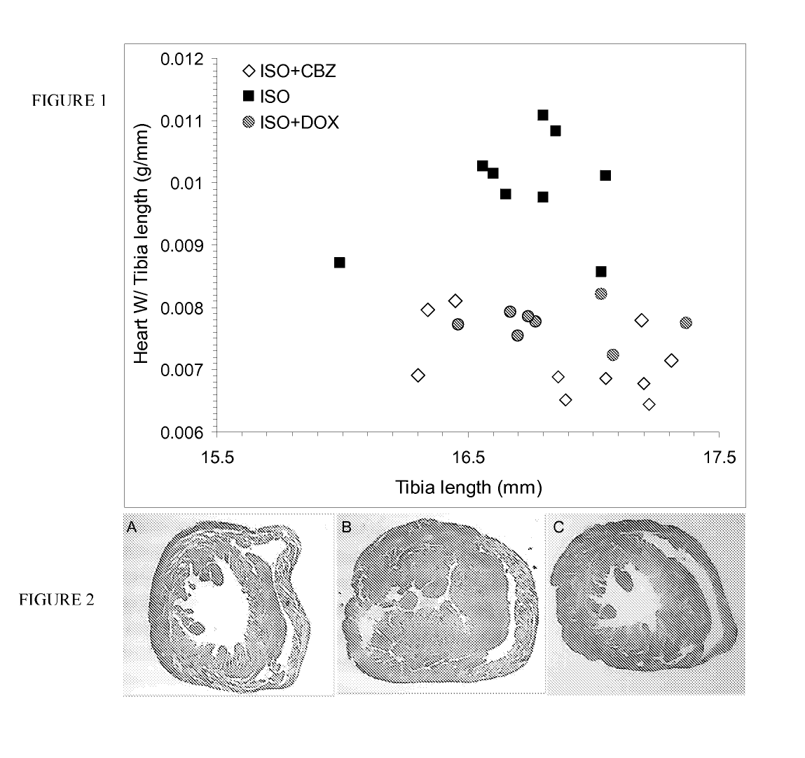

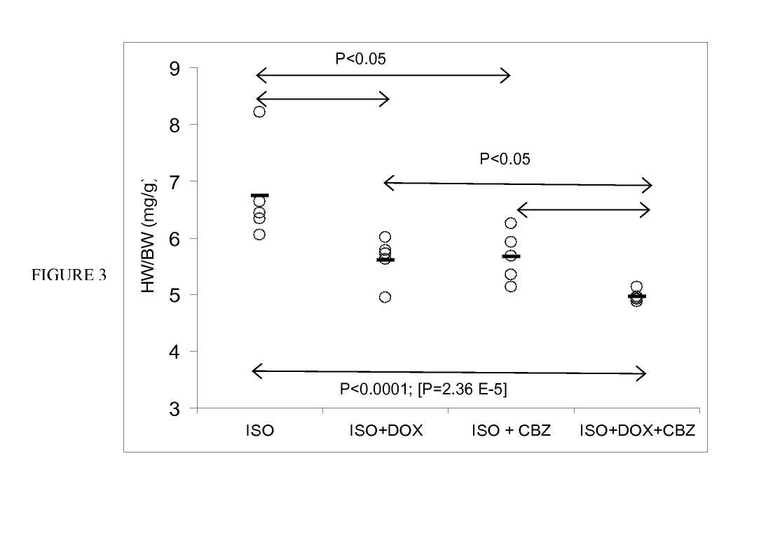

Carbamazepine is beneficial in the treatment of cardiac hypertrophy. FIG. 1 is a graph of the comparative effects of doxycycline and carbamazepine on the Heart to Tibia length ratio. The graph illustrates mice that received carbamazepine in chow (diamonds) or doxycycline in water (circles) or no drug (squares). Carbamazepine and doxycycline significantly lower the Heart to Tibia length ratios. The histological cross sections of mice hearts of carbamazepine treated and untreated mice can be seen in the images of FIGS. 2A-2C. One way ANOVA carbamazepine vs. Control P-values are highly significant: (Heart to Body weights ratio) P<0.01 FIG. 3 and (Heart to Tibia length ratio) P<0.0001.

FIGS. 2A-2C are images of histological cross sections of mice hearts of carbamazepine treated and untreated mice. FIG. 2A is an image of the histological cross section of a wild type control mouse (C57BL/6J) Heart weight is 0.1305 grams; body weight is 26.3 grams. FIG. 2B is an image of the histological cross section of isoproterenol treated carbamazepine untreated mouse with a heart weight of 0.1800 grams and a body weight of 26.3 grams. FIG. 2C is an image of the histological cross section of isoproterenol and carbamazepine treated mouse with a heart weight of 0.1415 grams and a body weight of 26.3 grams. The carbamazepine untreated mouse exhibit a severe hypertrophy. The carbamazepine treated heart has a structure that is relatively well preserved suggesting that carbamazepine may improve heart performance and life expectancy under this condition comparatively to carbamazepine untreated mice.

FIG. 3 is a graph of the added therapeutic benefits of the combination of doxycycline and carbamazepine on isoproterenol induced cardiac hypertrophy. P-values are obtained from a one way ANOVA. The treatment lasted 10 days and doxycycline was given at 10 mg/mL in 7% sucrose water (in the isoproterenol+doxycycline and the isoproterenol+doxycycline+carbamazepine groups). Carbamazepine was given in chow at 0.25% (in the isoproterenol+carbamazepine and the isoproterenol+doxycycline+carbamazepine groups). The control group (isoproterenol) received regular chow and 7% sucrose water. Each circle is the Heart to Body weights ratio obtained for a mouse, while the dashes are the average for each group.

As shown in FIGS. 1 and 3, carbamazepine significantly decreased Heart to Tibia length ratio (p value <0.0001) and Heart to Body weights ratio (p value <0.01), respectively. Carbamazepine treatment also reduced the hypertrophic phenotype, as determined by examination of heart cross sections as seen in FIG. 2, suggesting that carbamazepine also improves heart performance and survival time when challenged with high doses of isoproterenol over a longer period of time. In addition, significantly lower heart rates were observed in isoproterenol+carbamazepine treated mice than in isoproterenol animals.

The combination of carbamazepine and doxycycline confer additional benefits and longer survival times. A shorter half-life for doxycycline when co-administered with carbamazepine was previous reported (22) and therefore, the concentration of doxycycline was increased to 10 mg/mL in 7% sucrose when given along with carbamazepine. Based on heart to body weight ratios, the combination of carbamazepine and doxycycline conferred a greater benefit than either carbamazepine or doxycycline alone as seen in FIG. 3. The combination of two drugs act via different cardiac hypertrophy-associated pathways and that targeting them both simultaneously resulted in a better therapeutic performance and result in a synergy effect.

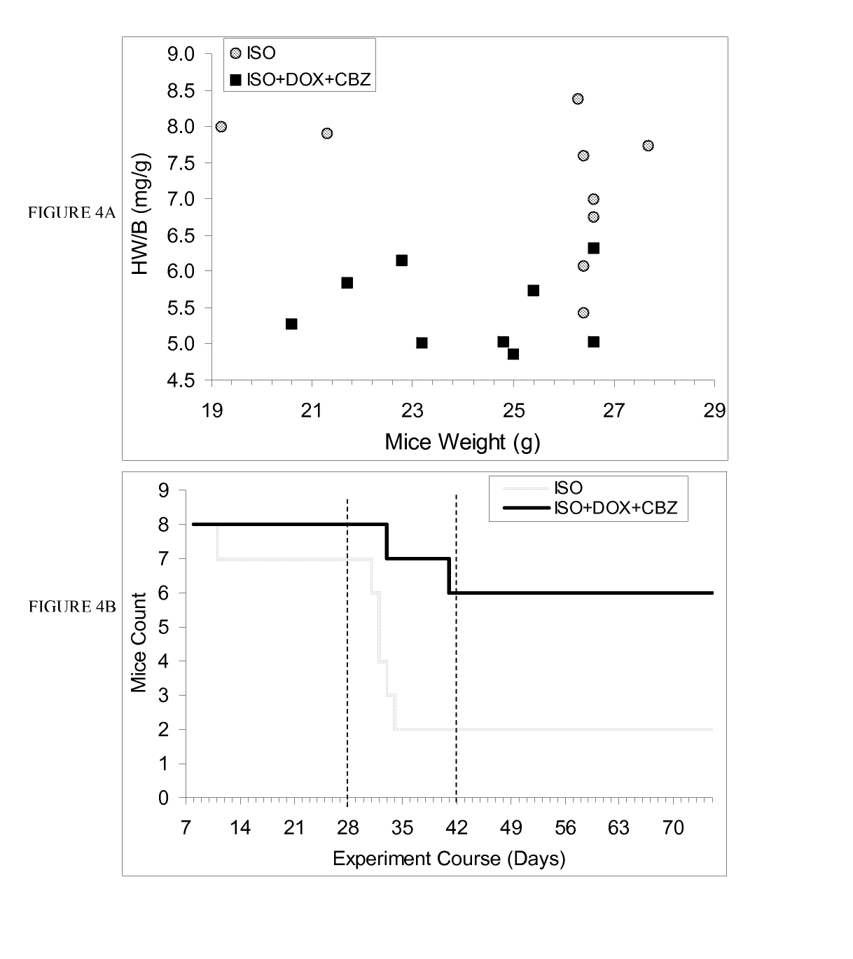

FIG. 4A is a graph of the heart size reading after death for one mouse sacrificed on day 7 in each group of 9 mice. FIG. 4B is a Kaplan survival curve. The combination therapy increases the survival rate 3-fold over the 75 day period. The treatment of doxycycline and carbamazepine translated into a substantial increase in survival time (i.e. three times longer than untreated mice over a 75 day period).

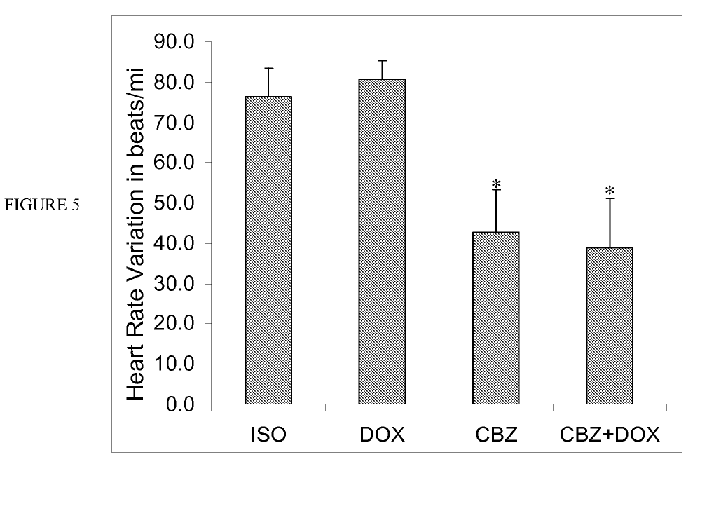

The heart rates of the mice were measured before induction of cardiac hypertrophy and after treatment on day 9, which was 1 day before they were sacrificed. Isoproterenol caused an observable increase in heart rate for each mouse to which it was administered, compared to measurements taken prior to isoproterenol treatment. FIG. 5 is a graph of the heart rate variation over course of experiment (average +SEM) of mice receiving isoproterenol or doxycycline and isoproterenol or carbamazepine and isoproterenol or isoproterenol and doxycycline and carbamazepine. Heart rates were measured before the experiment (t₀) and 1 day before the sacrifice. Each heart rate is the average of 3 measures. The One way ANOVA p-value is 0.007 and indicates differences in groups (n=5). The subsequent Newman-Keuls test led to the conclusion that groups can be classified as follows: isoproterenol=doxycycline≠carbamazepine=(carbamazepine and doxycycline), which indicates that the attenuation of the positive chronotropic effect induced by isoproterenol is mediated by carbamazepine. The maximum heart rate in the samples illustrated in FIG. 5 was 821 beats per minute for the ISO group versus 780 for the CH group that was treated with carbamazepine and doxycycline (p value <0.01, n=5 in each group). No clear alteration in heart rate was observed in mice treated with doxycycline alone, compared to those that received isoproterenol mice, indicating that the mechanism of action of doxycycline is independent of the β-adrenergic pathway.

Effects on Gene Expression Profile. In order to assess the effect of doxycycline on cardiac gene expression, microarray analysis was performed on normal mice (N), mice with isoproterenol-induced cardiac hypertrophy that were subsequently untreated (cardiac hypertrophy) or treated with doxycycline, carbamazepine, or doxycycline and carbamazepine (Combo). One mouse heart was used for each array, and was performed in triplicate, generating a total of 12 arrays. GeneSifter was used to perform RMA normalization, pairwise comparisons of averaged signal intensity values, and Student's t-test with Benjamini and Hochberg correction, and Spotfire was used to perform pairwise comparisons. A gene was considered as significantly altered in expression if the average fold-change value was at least 2.0, the fold-change for each individual replicate comparison was at least 1.5 and the corrected p value less than 0.05. Additionally, genes that were altered between any two N or cardiac hypertrophy samples were removed, as these alterations most likely represented normal variations between mice.

Based on these criteria, there were 779 genes that were significantly altered between N and CH mice as illustrated in TABLE 1. Of these 779 genes, 327 and 472 were altered in the reverse direction when mice were given doxycycline or the combination drug treatment, respectively. Only 1 gene was significantly altered, based on the stringent analysis criteria used, in mice treated with carbamazepine alone see also TABLE 1.

TABLE 1

| | | CH vs | CH vs | CH vs |
|---|---|---|---|---|
| | N vs CH | Dox | Cbz | Combo |
| Filtering Method | Number of Altered Genes | | | |
| Average FC (1.5-fold) | 3518 | 2929 | 709 | 3267 |
| Student's t test | 3037 | 2274 | 306 | 2688 |
| Correction | 2947 | 2034 | 1 | 2536 |
| Minus natural variability | 1345 | 994 | 1 | 1150 |
| Minus disease variability | — | 578 | 1 | 716 |
| Reproducible | 1059 | 497 | 1 | 627 |
| Average FC (2-fold) | 779 | 417 | 1 | 503 |
| Disease-specific | 779 | 327 | 1 | 472 |

Genes determined to be differentially expressed between the four samples types, based on statistical and filtering methods used. N represents normal mice, CH represents isoproterenol-treated mice; DOX represents mice treated with isoproterenol and doxycycline; CBZ represents mice treated with isoproterenol and carbamazepine; and Combo represents mice treated with isoproterenol and doxycycline and carbamazepine.

TABLE 2 illustrates genes that are significantly altered in mice treated with isoproterenol and doxycycline and carbamazepine (Combo), compared to mice given only isoproterenol. Average fold-change values regardless of level of significance are also shown for normal versus isoproterenol mice (CH) and isoproterenol mice compared to mice treated with either drug alone (doxycycline and carbamazepine). A copy of TABLE 2 is attached on computer readable media in the form of a compact disc (CD-R), filed in duplicate and the contents of which are incorporated herein. Average fold-change values regardless of level of significance are also shown for normal versus isoproterenol mice (CH) and isoproterenol mice compared to mice treated with either doxycycline or carbamazepine alone. The gene (G7e protein) encodes a viral capsid protein of otherwise unknown function (−2.2-fold).

TABLE 2

| | | | |
|---|---|---|---|
| CH | Combo | DOX | CBZ |

| | | | |
|---|---|---|---|
| GenBank ID | Gene name | Function | FC |

Genes Altered by CH and All Three Drug Regimens

| | | | | | | |
|---|---|---|---|---|---|---|
| AK016527 | Cadherin 13 (Cdh13) | Homophilic cell | −10.0 | 13.1 | 9.8 | 5.9 |
| | | adhesion | | | | |
| BI154147 | Heat shock protein, 84 kDa 1 | Stress response; | −2.6 | 6.3 | 8.7 | 3.7 |
| | (Hsp90ab1) | positive regulation of | | | | |
| | | nitric oxide | | | | |
| | | biosynthesis | | | | |
| BE995678 | Tumor rejection antigen gp96 | Stress response | −9.4 | 8.7 | 8.0 | 2.7 |
| | (Tra1); heat shock protein 90 kDa | | | | | |
| | beta (Grp94), member 1 (Hsp90b1) | | | | | |
| AK009897 | cDNA (similar to integrin beta 1 | Negative regulation of | −2.7 | 2.7 | 4.0 | 2.7 |
| | binding protein 3) | myoblast | | | | |
| | | differentiation | | | | |
| AF439339 | Kv channel-interacting protein 2a | Ion transport | −4.4 | 7.4 | 3.7 | 2.6 |
| | (Kcnip2) | | | | | |
| AB072269 | Desmoglein 2 (Dsg2) | Homophilic cell | −2.6 | 3.7 | 3.0 | 2.5 |
| | | adhesion; | | | | |
| | | cardiomyopathy | | | | |
| BB026304 | cDNA | Unknown | −3.8 | 5.8 | 4.1 | 2.5 |
| AW544889 | Karyopherin (importin) beta 1 | Protein import into | −2.8 | 6 | 3.6 | 2.3 |
| | (Kpnb1) | nucleus, docking | | | | |
| BB451134 | EST | Unknown | −1.8 | 8.2 | 7.5 | 2.2 |
| BB126796 | EST | Unknown | −1.8 | 6.9 | 6.5 | 2.2 |
| NM_007705 | Cold inducible RNA binding | ERK activation; anti- | −2.1 | 2.4 | 1.7 | 2.1 |
| | protein (Cirbp) | apoptosis | | | | |
| NM_008092 | GATA binding protein 4 (Gata4) | Transcription | −3.0 | 4 | 2.7 | 2.1 |
| | | regulation; heart | | | | |
| | | development | | | | |
| BB092799 | Nuclear factor IB (Nfib) | Negative regulation of | −1.7 | 3.3 | 4.0 | 2.0 |
| | | cell proliferation | | | | |
| NM_133249 | PPAR gamma coactivator-1beta | Mitochondrial | −2.9 | 3.4 | 4.4 | 2.0 |
| | protein (Ppargc1b) | metabolism; energy | | | | |
| | | balae | | | | |
| BB833716 | Tetratricopeptide repeat domain | Protein binding | −1.8 | 3.1 | 3.0 | 2.0 |
| | (Ttc3) | | | | | |
| AK018895 | Restin-like 2 (Rsnl2) | Unknown | −2.6 | 4.8 | 3.8 | 1.9 |
| AK014703 | Insulin degrading enzyme (Ide) | Proteolysis; inhibition | −2.4 | 2.3 | 2.1 | 1.9 |
| | | of insullin signaling | | | | |
| AW763746 | Jumonji domain containing 3 | Unknown | −2.7 | 4.6 | 5.0 | 1.9 |
| | (Jmjd3) | | | | | |
| BB175650 | Zi finger and BTB domain | DNA binding; protein | −2.0 | 2.8 | 2.3 | 1.9 |
| | containing 20 (Zbtb20) | binding | | | | |
| BB283973 | cDNA | Unknown | −3.3 | 4.5 | 4.1 | 1.9 |
| AI595932 | Myocyte enhaer factor 2C (Mef2c) | Transcription | −2.6 | 4.2 | 4.5 | 1.9 |
| | | regulation; cardiac | | | | |
| | | development | | | | |
| BB281000 | Cytoplasmic polyadenylation | Unknown | −1.7 | 3 | 2.6 | 1.8 |
| | element binding protein 3 (Cpeb3) | | | | | |
| BG965405 | B-cell translocation gene 2, anti- | Transcription | −2.5 | 2.8 | 2.2 | 1.8 |
| | proliferative (Btg2) | regulation | | | | |
| BC026793 | cDNA | Unknown | −2.5 | 2.8 | 2.5 | 1.8 |
| NM_008748 | Dual specificity phosphatase 8 | Signal transduction | −3.0 | 2.5 | 2.8 | 1.8 |
| | (Dusp8) | | | | | |
| BB376407 | Jumonji, AT rich interactive | Transcription | −1.9 | 2.1 | 2.6 | 1.8 |
| | domain 1A (Rbp2 like) (Jarid1a) | regulation | | | | |
| BG066667 | DNA segment, Chr 9, ERATO Doi | Negative regulation of | −2.4 | 3.5 | 3.3 | 1.8 |
| | 256, expressed | microtubule | | | | |
| | | depolymerization | | | | |
| BB794936 | Triple futional domain (PTPRF | Regulation of Rho | −1.7 | 2.7 | 3.0 | 1.7 |
| | interacting) (Trio) | protein signal | | | | |
| | | transduction | | | | |
| BM941198 | EST | Unknown | −1.9 | 2.3 | 3.2 | 1.7 |
| AW537707 | Actin, beta, cytoplasmic (Actb) | Structural constituent | −3.2 | 5 | 4.9 | 1.7 |
| | | of cytoskeleton | | | | |
| BB277041 | Methionine sulfoxide reductase B3 | Protein repair | −3.2 | 7.7 | 7.2 | 1.7 |
| | (Msrb3) | | | | | |
| BB550273 | Preimplantation protein 4 (Prei4) | Carbohydrate | −1.7 | 3.5 | 3.6 | 1.7 |
| | | metabolism | | | | |
| NM_013743 | Pyruvate dehydrogenase kinase 4 | Acetyl-CoA | 3.3 | −4.1 | −3.1 | 1.7 |
| | | biosynthesis from | | | | |
| | (Pdk4) | pyruvate; energy | | | | |
| | | production | | | | |
| NM_009762 | SET and MYND domain containing | Heart development | −2.9 | 3.2 | 2.7 | 1.7 |
| | 1 (Smyd1) | | | | | |
| NM_010302 | Guanine nucleotide binding protein, | G-protein coupled | −2.0 | 5.4 | 3.0 | 1.6 |
| | alpha 12 (Gna12) | receptor protein | | | | |
| | | signaling | | | | |
| D17577 | Kinesin-like protein (Kif1b) | Microtubule-based | −3.9 | 4.6 | 4.8 | 1.6 |
| | | movement | | | | |
| BB326749 | Metastasis suppressor 1 (Mtss1) | Cell motility; cell | −1.8 | 2 | 2.0 | 1.6 |
| | | adhesion; muscle | | | | |
| | | development | | | | |
| BB534971 | cDNA | Unknown | −3.5 | 2.3 | 2.9 | 1.6 |
| BE947961 | Sno, strawbeny notch homolog 1 | Negative regulation of | −2.4 | 2.8 | 3.0 | 1.6 |
| | (Sno1) | progression though | | | | |
| | | cell cycle | | | | |
| BM229539 | cDNA | Unknown | −1.6 | 2.3 | 2.1 | 1.6 |
| NM_007416 | Adrenergic receptor, alpha 1b | Blood vessel | −2.3 | 2.3 | 1.8 | 1.6 |
| | (Adra1b) | remodeling; regulation | | | | |
| | | of blood pressure and | | | | |
| | | heart contraction | | | | |
| AK012553 | Metallophosphoesterase domain | Metabolism | −1.5 | 2.5 | 2.0 | 1.6 |
| | containing 2 (Mpped2) | | | | | |
| M94335 | Thymoma viral proto-oogene 1 | Negative regulation of | −3.3 | 3.2 | 2.1 | 1.6 |
| | (Akt1) | apoptosis; germ cell | | | | |
| | | development | | | | |
| NM_026161 | C1q and tumor necrosis factor | Unknown | −2.1 | 2.3 | 1.5 | 1.5 |
| | related protein 4 (C1qtnf4) | | | | | |
| BQ175608 | EphrinB3 (Efnb3) | Development | −1.7 | 2.9 | 1.6 | 1.5 |
| NM_008424 | Potassium voltage-gated channel, | Epithelial cell | −4.6 | 3.6 | 1.5 | 1.5 |
| | Isk-related subfamily, member 1 | maturation; ion | | | | |
| | (Kcne1) | transport | | | | |
| NM_053110 | Glycoprotein (transmembrane) nmb | Cell adhesion | 2.9 | −2 | −2.0 | −1.5 |
| | (Gpnmb) | | | | | |
| NM_021400 | Proteoglycan 4 (megakaryocyte | Cartilage boundary | 6.8 | −3.8 | −3.3 | −1.5 |
| | stimulating factor, articular | lubrication | | | | |
| | superficial zone protein) (Prg4) | | | | | |
| AF282864 | Caer related gene-liver 1 (CRG-L1) | Metabolism | 4.9 | −3.3 | −3.0 | −1.5 |
| NM_008411 | CUB and zona pellucida-like | Substrate-bound cell | 3.2 | −2.4 | −1.7 | −1.5 |
| | domains 1 (Cuzd1) | migration, cell | | | | |
| | | attachment to substrate | | | | |
| AV293368 | Mcf.2 transforming sequee-like | Rho protein signal | 3.7 | −3.2 | −2.3 | −1.5 |
| | (Mcf21) | transduction | | | | |
| BC015260 | FK506 binding protein 5 (51 kDa) | Steroid signaling; | 16.3 | −8.9 | −4.5 | −1.6 |
| | (Fkbp5) | protein folding | | | | |
| NM_030612 | Nuclear factor of kappa light | Transcription | 3.9 | −3.4 | −2.7 | −1.6 |
| | polypeptide gene enhaer in B-cells | regulation; | | | | |
| | inhibitor, zeta (Nfkbiz) | inflammation | | | | |
| NM_007876 | Dipeptidase 1 (Dpep1) | Metabolism | 2.6 | −2.6 | −2.3 | −1.6 |
| BE630073 | EST | Unknown | 2.1 | −2.1 | −2.0 | −1.6 |
| NM_026835 | Membrane-spanning 4-domains, | Signal transduction | 6.4 | −3.6 | −2.3 | −1.6 |
| | subfamily A, member 6d (Ms4a6d) | | | | | |
| BC002148 | Fatty acid binding protein 4, | Cytokine production; | 1.8 | −2.3 | −2.1 | −1.6 |
| | adipocyte (Fabp4) | inflammation | | | | |
| X14607 | Lipocalin 2 (Lcn2) | Vascular remodeling; | 27.7 | −16.6 | −13.3 | −1.6 |
| | | apoptosis | | | | |
| BC011229 | Flavin containing monooxygenase 1 | Electron transport | 2.2 | −2 | −2.0 | −1.6 |
| | (Fmo1) | | | | | |
| NM_009841 | CD14 antigen (CD14) | Inflammation; I- | 2.2 | −2.3 | −2.0 | −1.6 |
| | | kappaB kinase/NF- | | | | |
| | | kappaB cascade; one- | | | | |
| | | half of LPS receptor | | | | |
| | | (with TLR4) | | | | |
| BG075321 | cDNA | Unknown | 2.2 | −2.6 | −2.5 | −1.6 |
| AV032095 | EST | Unknown | 2.1 | −2.3 | −1.8 | −1.6 |
| AK020831 | A disintegrin-like and | Proteolysis | 2.3 | −2.2 | −1.8 | −1.6 |
| | metallopeptidase (reprolysin type) | | | | | |
| | with thrombospondin (ADAMTS)- | | | | | |
| | like 2 (Adamtsl2) | | | | | |
| AV321547 | Decorin (Dcn) | Extracellular matrix | 2.9 | −2.1 | −2.0 | −1.6 |
| | | organization | | | | |
| AV228493 | Interleukin-1 receptor-associated | Cytokine and | 3.5 | −2.5 | −2.1 | −1.6 |
| | kinase 3 (Irak3) | chemokine mediated | | | | |
| | | signaling pathway; | | | | |
| | | apoptosis | | | | |
| BB831146 | CCAATenhaer binding protein | Transcription | 7.4 | −5.1 | −4.1 | −1.6 |
| | (CEBP), delta (Cebpd) | regulation | | | | |
| BC027310 | Fc fragment of IgG, low affinity | Immune response | 2.6 | −2.8 | −2.7 | −1.6 |
| | IIIa, receptor (Fcrl3) | | | | | |
| BB035924 | C-type lectin domain family 1, | Cell adhesion | 3.1 | −3.6 | −2.2 | −1.6 |
| | member a (Clec1a) | | | | | |
| NM_033075 | G7e protein | Viral capsid | 2.7 | −2.8 | −2.2 | −1.6 |
| BC002065 | Serine (or cysteine) peptidase | Apoptosis; immune | 3.2 | −3.8 | −3.6 | −1.7 |
| | inhibitor, clade A, member 3G | response | | | | |
| | (Serpina3g) | | | | | |
| AI117633 | TRAF2 and K interacting kinase | Signal transduction | 3.4 | −3.6 | −3.4 | −1.7 |
| | (Tnik) | | | | | |
| BC003788 | Purine-nucleoside phosphorylase | Nucleobase, | 2.4 | −2.4 | −2.0 | −1.7 |
| | (Pnp) | nucleoside, nucleotide | | | | |
| | | and nucleic acid | | | | |
| | | metabolism | | | | |
| NM_007746 | Mitogen activated protein kinase | Cell cycle regulation | 2.7 | −2.6 | −3.0 | −1.7 |
| | kinase kinase 8 (Map3k8) | | | | | |
| NM_011019 | Oostatin receptor (Osmr) | Inflammation; | 5.1 | −2.9 | −2.6 | −1.7 |
| | | connective tissue | | | | |
| | | production; | | | | |
| | | extracellular matrix | | | | |
| | | turnover | | | | |
| AW552579 | cDNA | Unknown | 2.6 | −2.7 | −2.4 | −1.7 |
| NM_010819 | C-type (calcium dependent, | Cell adhesion; immune | 10.8 | −3.6 | −2.7 | −1.7 |
| | carbohydrate recognition domain) | response | | | | |
| | lectin, superfamily member 8 | | | | | |
| | (Clecsf8) | | | | | |
| AK008807 | cDNA | Unknown | 4.5 | −2.9 | −2.7 | −1.7 |
| BE956710 | cDNA | Unknown | 3.7 | −3.9 | −4.4 | −1.7 |
| NM_009675 | Amine oxidase, copper containing 3 | Cell adhesion; | 2.2 | −2.1 | −2.4 | −1.8 |
| | (Aoc3) | lymphocyte | | | | |
| | | recirculation | | | | |
| AF047838 | Calcium-sensitive chloride | Ion transport | 4.4 | −3.7 | −3.6 | −1.8 |
| | conductae protein-1 (Clca1) | | | | | |
| NM_007781 | Colony stimulating factor 2 | Cytokine and | 4.1 | −4.3 | −2.9 | −1.8 |
| | receptor, beta 2, low-affinity | chemokine mediated | | | | |
| | (granulocyte-macrophage) | signaling pathway | | | | |
| | (Csf2rb2) | | | | | |
| NM_008489 | Lipopolysaccharide binding protein | Defense response to | 3.8 | −3.4 | −3.4 | −1.8 |
| | (Lbp) | bacteria | | | | |
| AW536690 | Procollagen, type IV, alpha 1 | Cell adhesion | 1.6 | −2.5 | −2.4 | −1.8 |
| | (Col4a1) | | | | | |
| AI447357 | ESTs | Unknown | 2.5 | −2.8 | −2.5 | −1.9 |
| BC021378 | NADPH oxidase 4 (Nox4) | Electron transport; | 4.3 | −3.4 | −3.2 | −1.9 |
| | | superoxide release | | | | |
| NM_011315 | Serum amyloid A 3 (Saa3) | Acute-phase response | 4.7 | −4.6 | −4.1 | −2.0 |
| AK012898 | cDNA | Unknown | 3.3 | −3.1 | −2.9 | −2.0 |
| AF108501 | Ca(2+)-sensitive chloride channel 2 | Chloride transport; | 7.4 | −5.7 | −5.1 | −2.0 |
| | (Clca2) | apoptosis | | | | |
| M65143 | Lysyl oxidase (Lox) | Connective tissue | 7.4 | −3.9 | −2.7 | −2.1 |
| | | modeling | | | | |
| BC027314 | G7e protein | Viral capsid | 3.2 | −3.7 | −3.1 | −2.2 |
| NM_007398 | Adenosine deaminase (Ada) | Nucelic acid | 2.1 | −2.3 | −2.4 | −2.2 |
| | | metabolism; immune | | | | |
| | | response | | | | |
| BC019553 | cDNA | Unknown | 2.7 | −3.4 | 1.7 | −2.2 |
| BB241535 | Cytokine inducible SH2-containing | Regulation of cell | 6.0 | −5.3 | −3.5 | −2.3 |
| | protein 3 (Socs3) | growth; regulation of | | | | |
| | | cytokine signaling | | | | |
| NM_009252 | Serine protease inhibitor 2-2 (Spi2- | Acute-phase response | 40.5 | −12.7 | −9.7 | −2.3 |
| | 2) (Serpin3n) | | | | | |
| BB831725 | Cytokine inducible SH2-containing | Regulation of cell | 8.2 | −7.9 | −4.9 | −2.4 |
| | protein 3 (Socs3) | growth; negative | | | | |
| | | regulation of insulin | | | | |
| | | signaling | | | | |
| NM_010728 | Lysyl oxidase (Lox) | Crosslinking of | 11.1 | −5.4 | −3.0 | −2.4 |
| | | collagen and elastin | | | | |
| BG862223 | Calcium/calmodulin-dependent | G1/S transition of | 4.9 | −4.5 | −4.0 | −2.8 |
| | protein kinase II, beta (Camk2b) | mitotic cell cycle | | | | |
| BG297038 | cDNA | Unknown | 3.2 | −4.4 | −3.3 | −2.9 |

Genes Altered by CH, DOX Treatment, and DOX + CBZ Ttreatment but not CBZ alone

| | | | | | | |
|---|---|---|---|---|---|---|
| NM_007470 | Apolipoprotein D (Apod) | Transport | 6.4 | −2.8 | −3.4 | NC |
| NM_009994 | Cytochrome P450, 1b1, | Metabolism | 6.4 | −4.3 | −3.2 | NC |
| | benz(a)anthracene inducible | | | | | |
| | (Cyp1b1) | | | | | |
| BG066678 | mVL30-1 retroelement mRNA | Unknown | 6.6 | −6.6 | −4.2 | NC |
| | sequee | | | | | |
| NM_019930 | RAN binding protein 9 (Ranbp9), | Signal transduction | 2.2 | −2.1 | −2.3 | NC |
| | mRNA. | | | | | |
| AB006361 | Prostaglandin D synthetase (Ptdgs) | Prostaglandin | 3.1 | −2.4 | −2.3 | NC |
| | | biosynthesis; muscle | | | | |
| | | contraction relaxation | | | | |
| BB667786 | Actin binding LIM protein family, | Cytoskeleton | 1.8 | −2.7 | −2.4 | NC |
| | member 3 (Ablim3) | organization and | | | | |
| | | biogenesis | | | | |
| NM_009647 | Adenylate kinase 4 (Ak4) | Nucleic acid | −2.1 | 2 | 2.2 | NC |
| | | metabolism | | | | |
| AV023312 | ADP-ribosylation factor 2 (Arf2) | ER to Golgi vesicle- | −2.8 | 2.3 | 2.5 | NC |
| | | mediated transport | | | | |
| BC013477 | Alcohol dehydrogenase 1, complex | Metabolism | 3.9 | −3.5 | −3.4 | NC |
| | (Adh1) | | | | | |
| AI256465 | Alpha-2-HS-glycoprotein (Ahsg) | Protease inhibition | 4.0 | −2.5 | −2.7 | NC |
| AV326938 | Amyotrophic lateral sclerosis 2 | Unknown | 3.0 | −2.3 | −1.6 | NC |
| | (juvenile) chromosome region, | | | | | |
| | candidate 13 (Als2cr13) | | | | | |
| NM_007447 | Angiogenin, ribonuclease A family, | Angiogenesis | 2.5 | −2.7 | −2.1 | NC |
| | member 1 (Ang1) | | | | | |
| AI385586 | Angiogenin, ribonuclease A family, | Angiogenesis; | 3.2 | −2.9 | −2.1 | NC |
| | member 1 (Ang1) | development | | | | |
| AI385586 | Angiogenin, ribonuclease A family, | Angiogenesis; | 2.1 | −2 | −1.6 | NC |
| | member 1 (Ang1) | development | | | | |
| C79906 | Ankyrin repeat domain 47 | Unknown | 1.6 | −2.1 | −2.0 | NC |
| | (Ankrd47) | | | | | |
| BQ176992 | Apical protein, Xenopus laevis-like | Unknown | 2.4 | −2.2 | −1.7 | NC |
| | (Apx1) | | | | | |
| AW542672 | Arrestin domain containing 2 | Unknown | 4.6 | −4.2 | −3.5 | NC |
| | (Arrdc2) | | | | | |
| BC011080 | Aryl hydrocarbon receptor nuclear | Protein import into | 5.0 | −4.8 | −5.1 | NC |
| | translocator-like (Arnt1) | nucleus; signaling | | | | |
| BB079486 | AT rich interactive domain 5B | Transcription | 1.8 | −2.1 | −2.0 | NC |
| | (Mrf1 like) (Arid5b) | regulation | | | | |
| C78762 | ATP synthase, H+ transporting, | ATP synthesis coupled | −3.1 | 2.7 | 1.7 | NC |
| | mitochondrial F1 complex, alpha | proton transport | | | | |
| | subunit, isoform 1 (Atp5a1) | | | | | |
| BC025618 | ATPase, Na+/K+ transporting, | Ion transport | −1.7 | 2.3 | 2.4 | NC |
| | alpha 1 polypeptide (Atp1a1) | | | | | |
| BC025618 | ATPase, Na+/K+ transporting, | Ion transport; blood | −2.0 | 2.6 | 2.7 | NC |
| | alpha 1 polypeptide (Atp1a1) | pressure regulation; | | | | |
| | | cardiac contraction | | | | |
| BB305534 | ATP-binding cassette, sub-family A | Phagocytosis, | 3.7 | −2.2 | −1.9 | NC |
| | (ABC1), member 1 (Abca1) | engulfment | | | | |
| NM_011920 | ATP-binding cassette, sub-family G | Transport | 2.5 | −2.1 | −1.7 | NC |
| | (WHITE), member 2 (Abcg2) | | | | | |
| U73626 | ATP-sensitive potassium channel | Ion transport | −1.6 | 2.7 | 1.8 | NC |
| | subunit (Kir6.2) (Kcnj11) | | | | | |
| AW321975 | Transglutaminase 2, C polypeptide | Proteolysis; G-protein | 2.0 | −2.7 | −1.8 | NC |
| | (Tgm2) | signaling, coupled to | | | | |
| | | IP3 second messenger | | | | |
| | | (phospholipase C | | | | |
| | | activating) | | | | |
| NM_009760 | BCL2/adenovirus E1B interacting | Apoptosis | 4.1 | −2.2 | −2.0 | NC |
| | protein 1, NIP3 (Bnip3) | | | | | |
| BM228788 | Bcl2-like (Bcl211) | Anti-apoptosis | 2.7 | −2.2 | −1.9 | NC |
| M28739 | Beta-tubulin gene M-beta-2 | Microtubule-based | −2.1 | 2.3 | 1.7 | NC |
| | (Tubb2a) | movement | | | | |
| NM_007607 | Carbonic anhydrase 4 (Car4) | Cell differentiation | 3.1 | −4.6 | −5.2 | NC |
| NM_007607 | Carbonic anhydrase 4 (Car4) | Cell differentiation | 2.8 | −4.2 | −4.3 | NC |
| BB205662 | Casitas B-lineage lymphoma b | Immune response; T | 2.6 | −2.5 | −2.1 | NC |
| | (Cblb) | cell activation | | | | |
| AW545867 | Casitas B-lineage lymphoma b | Immune response | 3.1 | −2.1 | −2.1 | NC |
| | (Cblb) | | | | | |
| AV276986 | Casitas B-lineage lymphoma b | Immune response; T | 2.0 | −2.1 | −1.7 | NC |
| | (Cblb) | cell activation | | | | |
| BC025116 | Cbp/p300-interacting transactivator, | Transcription | −2.6 | 2.4 | 2.0 | NC |
| | with Glu/Asp-rich carboxy-terminal | regulation | | | | |
| | domain, 4 (Cited4) | | | | | |
| NM_009883 | CCAATenhaer binding protein | Transcription | 2.3 | −2.2 | −1.9 | NC |
| | (CEBP), beta (Cebpb) | regulation; anti- | | | | |
| | | apoptosis | | | | |
| NM_053094 | CD163 antigen (CD163) | Acute-phase response; | 3.6 | −2.9 | −2.1 | NC |
| | | inflammation | | | | |
| NM_054042 | CD248 antigen, endosialin (CD248) | Stromal fibroblast | −1.6 | 2 | 1.7 | NC |
| | | marker | | | | |
| AK002762 | CD99 antigen (CD99) | Cell adhesion | −1.6 | 2.3 | 1.6 | NC |
| BF682848 | cDNA | Unknown | 10.6 | −7.5 | −5.7 | NC |
| BF682848 | cDNA | Unknown | 21.4 | −11.4 | −5.2 | NC |
| AI607873 | cDNA | Unknown | 5.5 | −5.4 | −4.0 | NC |
| BC020080 | cDNA | Unknown | 2.9 | −3.1 | −3.4 | NC |
| NM_133898 | cDNA | Unknown | 3.2 | −3.1 | −3.3 | NC |
| BF719154 | cDNA | Unknown | 7.0 | −6.9 | −3.2 | NC |
| AU018141 | cDNA | Unknown | 7.8 | −6.7 | −3.2 | NC |
| BM117672 | cDNA | Unknown | 4.6 | −3.2 | −3.2 | NC |
| AA939619 | cDNA | Unknown | 3.1 | −2.5 | −3.1 | NC |
| BG276629 | cDNA | Unknown | 3.7 | −3.3 | −3.1 | NC |
| BB829165 | cDNA | Unknown | 3.2 | −3.6 | −3.1 | NC |
| BC004065 | cDNA | Unknown | 2.9 | −2.2 | −3.1 | NC |
| AK009753 | cDNA | Unknown | 5.4 | −3.4 | −3.0 | NC |
| AV365503 | cDNA | Unknown | 2.8 | −4.4 | −3.0 | NC |
| BE634869 | cDNA | Unknown | 5.2 | −4.6 | −2.8 | NC |
| AV365503 | cDNA | Unknown | 3.1 | −4.2 | −2.8 | NC |
| BC027342 | cDNA | Unknown | 2.4 | −2.2 | −2.8 | NC |
| BF466929 | cDNA | Unknown | 2.3 | −2.8 | −2.8 | NC |
| BG071024 | cDNA | Unknown | 2.7 | −2.7 | −2.8 | NC |
| BB200607 | cDNA | Unknown | 2.7 | −3.7 | −2.7 | NC |
| BI683916 | cDNA | Unknown | 1.9 | −2.5 | −2.6 | NC |
| AV369812 | cDNA | Unknown | 3.2 | −2.9 | −2.5 | NC |
| BG065702 | cDNA | Golgi to plasma | 2.5 | −2.6 | −2.5 | NC |
| | | membrane protein | | | | |
| | | transport | | | | |
| BB795572 | cDNA | Unknown | 5.3 | −3.2 | −2.3 | NC |
| BB787946 | cDNA | Unknown | 2.5 | −3.2 | −2.3 | NC |
| AI788755 | cDNA | Unknown | 2.1 | −2.2 | −2.3 | NC |
| BB258019 | cDNA | Unknown | 2.3 | −2.2 | −2.3 | NC |
| BB398891 | cDNA | Unknown | 2.4 | −2.4 | −2.2 | NC |
| BE956940 | cDNA | Unknown | 2.5 | −3.9 | −2.2 | NC |
| BB769119 | cDNA | Unknown | 2.2 | −2.7 | −2.2 | NC |
| BB431047 | cDNA | Unknown | 2.1 | −2.1 | −2.2 | NC |
| BQ174442 | cDNA | Protein biosynthesis | 2.7 | −2.3 | −2.2 | NC |
| BB038506 | cDNA | Unknown | 3.9 | −4.3 | −2.1 | NC |
| BC003209 | cDNA | Unknown | 2.3 | −2.1 | −2.1 | NC |
| BB248249 | cDNA | Unknown | 2.3 | −2.2 | −2.1 | NC |
| AV228737 | cDNA | Unknown | 2.0 | −2.1 | −2.1 | NC |
| AI790538 | cDNA | Unknown | 3.1 | −2.6 | −2.0 | NC |
| BB408123 | cDNA | Unknown | 2.2 | −2.6 | −2.0 | NC |
| BG919470 | cDNA | Unknown | 2.2 | −2 | −2.0 | NC |
| BG073457 | cDNA | Unknown | 1.7 | −2 | −1.9 | NC |
| BB098431 | cDNA | Unknown | 3.4 | −2.1 | −1.8 | NC |
| AV234245 | cDNA | Unknown | 2.6 | −2.4 | −1.8 | NC |
| BI689897 | cDNA | Unknown | 1.7 | −2.1 | −1.8 | NC |
| AK005293 | cDNA | Unknown | 1.8 | −2 | −1.8 | NC |
| AV084904 | cDNA | Unknown | 3.2 | −2.9 | −1.7 | NC |
| BM215139 | cDNA | Unknown | 1.8 | −2 | −1.7 | NC |
| BB447627 | cDNA | Unknown | 2.0 | −2 | −1.6 | NC |
| BB272245 | cDNA | Unknown | 2.3 | −2.3 | −1.6 | NC |
| AV293532 | cDNA | Unknown | 1.7 | −2 | −1.5 | NC |
| AK020162 | cDNA | Unknown | −1.7 | 3 | 1.7 | NC |
| BB006809 | cDNA | Unknown | −1.9 | 2.3 | 2.1 | NC |
| BB627097 | cDNA | Unknown | −1.6 | 2.1 | 2.4 | NC |
| AK013448 | cDNA | Unknown | −2.9 | 2.3 | 2.6 | NC |
| BB748887 | cDNA | Unknown | −2.4 | 3.1 | 3.9 | NC |
| BC002200 | cDNA | Unknown | −2.0 | 3.8 | 4.8 | NC |
| BB550183 | cDNA (D site albumin promoter | Transcription | −11.5 | 14.5 | 12.0 | NC |
| | binding protein, Dbp) | regulation; cricadian | | | | |
| | | rhythm | | | | |
| NM_007752 | Ceruloplasmin (Cp) | Ion transport | 2.7 | −2.5 | −1.7 | NC |
| BC025169 | ChaC, cation transport regulator- | Unknown | 2.9 | −3 | −2.6 | NC |
| | like 1 (Chac1) | | | | | |
| BC002073 | Chemokine (C—C motif) ligand 6 | Chemotaxis; immune | 3.2 | −3 | −1.9 | NC |
| | (Cc16) (MRP-1) | response | | | | |
| NM_009892 | Chitinase 3-like 3 (Chi313) | Inflammation | 4.1 | −4.2 | −4.1 | NC |
| AY065557 | Chitinase 3-like 3; chitinase 3-like 4 | Inflammatory response | 1.9 | −2.4 | −2.4 | NC |
| AA210377 | Chloride intracellular channel 5 | Chloride transport | 1.8 | −2.2 | −1.7 | NC |
| | (Circ5) | | | | | |
| NM_013490 | Choline kinase (Chk) | Phosphatidylcholine | 2.0 | −2.1 | −1.9 | NC |
| | | biosynthesis | | | | |
| NM_009881 | Chromodomain protein, Y | Chromatin assembly or | 3.0 | −2.8 | −2.0 | NC |
| | chromosome-like (Cdyl) | disassembly | | | | |
| AW060797 | Coiled-coil domain containing 85A | Unknown | 2.0 | −2.6 | −2.0 | NC |
| | (Ccdc85a) | | | | | |
| BB739754 | Connector enhaer of kinase | Ras/Rho protein signal | 4.4 | −4.4 | −3.6 | NC |
| | suppressor of Ras 1 (Cnksr1) | transduction | | | | |
| M63801 | Connexin 43 (alpha-1 gap jution) | Regulation of heart | −3.9 | 5.3 | 3.6 | NC |
| | (Gja1) | contraction rate; | | | | |
| | | vascualr remodeling | | | | |
| NM_011779 | Coronin, actin binding protein 1C | Cytoskeletal | −2.1 | 2.3 | 2.1 | NC |
| | (Coro1c) | organization and | | | | |
| | | biosynthesis | | | | |
| AF030636 | CXC chemokine (angie2) (Cxcl13) | Chemotaxis; | 2.1 | −2.6 | −2.2 | NC |
| | | inflammation | | | | |
| AK015150 | CXXC finger 5 (Cxxc5) | Unknown | −2.4 | 2.5 | 2.1 | NC |
| BB538325 | Cyclin D1 (Ccnd1) | Regulation of | −3.3 | 2.6 | 2.7 | NC |
| | | progression through | | | | |
| | | cell cycle | | | | |
| NM_007635 | Cyclin G2 (Ccng2) | Cell cycle regulation | 3.4 | −3.8 | −3.1 | NC |
| U95826 | Cyclin G2 (Ccng2) | Cell cycle regulation | 2.2 | −2.5 | −2.5 | NC |
| AK007630 | Cyclin-dependent kinase inhibitor | Cell cycle arrest | 14.6 | −8.6 | −6.5 | NC |
| | 1A (p21) (Cdkn1a) | | | | | |
| J02583 | Cysteine proteinase cathepsin L | Lysosomal proteion | 2.6 | −2.1 | −2.1 | NC |
| | (Ctsl) | degradation; critical for | | | | |
| | | cardiac morphology | | | | |
| | | and fution | | | | |
| AF332060 | Cytochrome b-5 reductase (Cyb5r3) | Electron transport | −2.2 | 2.9 | 1.7 | NC |
| BM899392 | Cytoglobin (Cygb) | Response to oxidative | −1.8 | 2.2 | 1.8 | NC |
| | | stress | | | | |
| M12481 | Cytoplasmic beta-actin (Actb) | Cytoskeletal | −2.5 | 2.9 | 3.1 | NC |
| | | constituent | | | | |
| BC018323 | D site albumin promoter binding | Transcription | −11.7 | 15.2 | 10.2 | NC |
| | protein (Dbp) | regulation; cricadian | | | | |
| | | rhythm | | | | |
| BB667395 | Dehydrogenase E1 and | Glycolysis | 2.8 | −2.6 | −2.1 | NC |
| | transketolase domain containing 1 | | | | | |
| | (Dhtkd1) | | | | | |
| AI647687 | Dipeptidase 1 (Dpep1) | Proteolysis | 2.9 | −2.8 | −2.0 | NC |
| AK017926 | DNA-damage-inducible transcript 4 | Hypoxic stress | 7.4 | −6.2 | −5.6 | NC |
| | (Ddit4) | response; cell growth | | | | |
| AK012530 | Dual specificity phosphatase 4 | Signal transduction | 3.3 | −2.8 | −2.4 | NC |
| | (Dusp4) | | | | | |
| NM_007897 | Early B-cell factor (Ebf1) | Transcription | −1.7 | 2.3 | 2.1 | NC |
| | | regulation; | | | | |
| | | development | | | | |
| BM120053 | Ectodermal-neural cortex 1 (E1) | Proteolysis; | 2.8 | −2.3 | −2.0 | NC |
| | | development | | | | |
| BM120053 | Ectodermal-neural cortex 1 (E1) | Proteolysis; | 2.8 | −2 | −1.9 | NC |
| | | development | | | | |
| AV117919 | Ectonucleoside triphosphate | G-protein coupled | 3.1 | −3.3 | −2.1 | NC |
| | diphosphohydrolase 1 (Entpd1) | receptor protein | | | | |
| | | signaling | | | | |
| BC017134 | EGF, latrophilin seven | G-protein coupled | 1.9 | −2.2 | −2.0 | NC |
| | transmembrane domain containing | receptor protein | | | | |
| | 1 (Eltd1) | signaling | | | | |
| NM_133222 | EGF, latrophilin seven | G-protein coupled | 1.8 | −2 | −1.7 | NC |
| | transmembrane domain containing | receptor protein | | | | |
| | 1 (Eltd1) | signaling | | | | |
| BB133079 | Endothelial differentiation | Angiogenesis | 2.5 | −2.6 | −2.4 | NC |
| | sphingolipid G-protein-coupled | | | | | |
| | receptor 1 (Edg1) | | | | | |
| NM_007945 | Epidermal growth factor receptor | Proteolysis; enhaement | 2.8 | −2.2 | −1.8 | NC |
| | pathway substrate 8 (Eps8) | of mitogenic signals | | | | |
| NM_133753 | ERBB receptor feedback inhibitor 1 | Stress-activated protein | 3.0 | −3.2 | −2.7 | NC |
| | (Errifi1) | kinase signaling | | | | |
| | | pathway | | | | |
| BG092512 | EST | Unknown | 4.8 | −4.2 | −2.9 | NC |
| BM219553 | EST | Unknown | 3.0 | −3.4 | −2.6 | NC |
| BM245060 | EST | Unknown | 3.2 | −2.4 | −2.3 | NC |
| BB312992 | EST | Unknown | 1.9 | −3.1 | −2.1 | NC |
| BB219003 | EST | Unknown | 2.4 | −2.5 | −2.1 | NC |
| BB535847 | EST | Unknown | 1.7 | −2.8 | −2.0 | NC |
| AI467657 | EST | Unknown | 12.8 | −3.2 | −1.9 | NC |
| AW208574 | EST | Unknown | 1.6 | −2 | −1.9 | NC |
| BF780807 | EST | Unknown | 2.0 | −2 | −1.9 | NC |
| AW123929 | EST | Unknown | 1.7 | −2.3 | −1.8 | NC |
| BB096843 | EST | Unknown | 2.2 | −2.1 | −1.8 | NC |
| BE630303 | EST | Unknown | 2.7 | −2.4 | −1.8 | NC |
| BB109391 | EST | Unknown | 1.9 | −2.3 | −1.8 | NC |
| BG068705 | EST | Unknown | 2.9 | −2.1 | −1.7 | NC |
| BB127176 | EST | Unknown | 2.5 | −2.4 | −1.7 | NC |
| BB236747 | EST | Unknown | 1.8 | −2.3 | −1.7 | NC |
| AA419994 | EST | Unknown | 8.5 | −2.6 | −1.7 | NC |
| BE553782 | EST | Unknown | 1.6 | −2.1 | −1.6 | NC |
| AI467657 | EST | Unknown | 5.6 | −2.6 | −1.6 | NC |
| BQ176399 | EST | Unknown | 1.6 | −2 | −1.6 | NC |
| BE687858 | EST | Unknown | 3.5 | −2.3 | −1.5 | NC |
| AV032877 | EST | Unknown | 1.7 | −2 | −1.5 | NC |
| AI480750 | EST | Unknown | −2.9 | 2.4 | 1.7 | NC |
| BE852759 | EST | Unknown | −1.9 | 2.1 | 1.7 | NC |
| BB476794 | EST | Unknown | −1.7 | 2.1 | 1.9 | NC |
| BB069531 | EST | Unknown | −1.7 | 2 | 2.1 | NC |
| BB335101 | EST | Unknown | −2.4 | 2.5 | 2.2 | NC |
| AV318727 | EST | Unknown | −1.6 | 2.3 | 2.6 | NC |
| BB374879 | EST | Unknown | −1.7 | 2.8 | 2.7 | NC |
| BE685667 | ESTs | Unknown | 4.6 | −3.3 | −3.2 | NC |
| BE630363 | ESTs | Unknown | 4.2 | −3.8 | −3.1 | NC |
| BM246377 | ESTs | Unknown | 2.6 | −2.4 | −1.9 | NC |
| BG067678 | ESTs | Unknown | −2.5 | 2.5 | 2.6 | NC |
| AK003461 | Ets variant gene 5 (Etv5) | Transcription | 1.7 | −2.4 | −2.0 | NC |
| | | regulation; organ | | | | |
| | | morphogenesis | | | | |
| AK004726 | Extra cellular link domain- | Glycosaminoglycan | 3.1 | −3.6 | −1.8 | NC |
| | containing 1 (Xkd1) | catabolism; cell | | | | |
| | | adhesion | | | | |
| BB503935 | F-box and leucine-rich repeat | Ubiquitin cycle | −1.9 | 3 | 1.9 | NC |
| | protein 13 (Fbxl13) | | | | | |
| AK012109 | F-box and leucine-rich repeat | Ubiquitin cycle | 1.7 | −2.5 | −2.0 | NC |
| | protein 20 (Fbxl20) | | | | | |
| AV120094 | F-box and leucine-rich repeat | Ubiquitin cycle | 2.0 | −2.2 | −1.5 | NC |
| | protein 20 (Fbxl20) | | | | | |
| NM_133765 | F-box only protein 31 (Fboxo31) | Unknown | 2.5 | −2.5 | −2.4 | NC |
| NM_026346 | F-box only protein 32 (Fbxo32) | Ubiquitin cycle | 2.5 | −4 | −4.7 | NC |
| AF441120 | F-box only protein 32 (Fbxo32) | Ubiquitin cycle | 2.4 | −3.5 | −3.3 | NC |
| AV338062 | F-box-WD40 repeat protein 6 | Ubiquitin cycle | −1.6 | 2.2 | 1.9 | NC |
| | (Fbxw6) | | | | | |
| AF391192 | F-box-WD40 repeat protein 6 | Ubiquitin cycle | −1.8 | 3 | 2.9 | NC |
| | (Fbxw6) | | | | | |
| NM_030614 | Fibroblast growth factor 16 (Fgf16) | Cell growth | −3.8 | 4.1 | 2.8 | NC |
| BB732903 | Fibroblast growth factor receptor 3 | Cell adhesion; | 3.2 | −2.4 | −2.1 | NC |
| | (Fgfr3) | MAPKKK cascade; | | | | |
| | | negative regulation of | | | | |
| | | cell proliferation | | | | |
| AI098139 | FK506 binding protein 5 (51 kDa) | Steroid signaling; | 5.8 | −4.6 | −4.1 | NC |
| | (Fkbp5) | protein folding | | | | |
| BM936480 | Flavin containing monooxygenase 2 | Oxygen and reactive | 4.6 | −3.1 | −2.7 | NC |
| | (Fmo2) | oxygen species | | | | |
| | | metabolism | | | | |
| BM245170 | Fos-like antigen 2 (Fosl2) | Transcription | 2.1 | −3.2 | −2.6 | NC |
| | | regulation | | | | |
| BB083808 | G protein-coupled receptor 116 | G-protein coupled | 2.1 | −2.7 | −2.0 | NC |
| | (Gprl16) | receptor protein | | | | |
| | | signaling | | | | |
| AF180518 | GABA-A receptor-associated | Vacuolar transport; | 3.3 | −2.5 | −3.6 | NC |
| | protein-like protein 1 (Gabarapl1) | autophagy | | | | |
| AF180518 | GABA-A receptor-associated | Autophagy | 3.2 | −2.7 | −3.1 | NC |
| | protein-like protein 1 (Gabarapl1) | | | | | |
| U10551 | Gem GTPase (Gem) | Calcium channel | 2.7 | −2.9 | −2.8 | NC |
| | | blockage | | | | |
| NM_010286 | Glucocorticoid-induced leucine | Anti-apoptosis; | 3.4 | −2.9 | −2.5 | NC |
| | zipper (Gilz) (Dsip1) | transcription regulation | | | | |
| U09114 | Glutamate-ammonia ligase (Glu1) | Glutamine biosyntehsis | 3.7 | −3.2 | −3.5 | NC |
| NM_008129 | Glutamate-cysteine ligase, modifier | Glutathione | 2.8 | −2.1 | −1.8 | NC |
| | subunit (Gclm) | biosynthesis | | | | |
| AK003305 | GPI-ahored HDL-binding protein 1 | Cholesterol transport | 1.6 | −2.3 | −1.8 | NC |
| | (Gpihbp1) | | | | | |
| AF162713 | Group V phospholipase A2 | Amplification of | −1.9 | 2.5 | 2.2 | NC |
| | (Pla2g5) | eicosanoid production | | | | |
| BM119226 | GTL2, imprinted maternally | Unknown | −1.5 | 2.6 | 1.7 | NC |
| | expressed untranslated mRNA | | | | | |
| | (Gtl2) | | | | | |
| BE136057 | Guanine deaminase (Gda) | Metabolism | 2.4 | −2.2 | −1.7 | NC |
| BQ031006 | Headcase homolog (Heca) | Unknown | 3.2 | −2.3 | −2.2 | NC |
| AI451467 | Heparan sulfate 2-O- | Heparan sulfate | −2.0 | 2.5 | 2.2 | NC |
| | sulfotransferase 1 (Hs2st1) | proteoglycan | | | | |
| | | biosynthesis, | | | | |
| | | polysaccharide chain | | | | |
| | | biosynthesis | | | | |
| BB822465 | Heterogeneous nuclear | Nucleotide binding | −2.7 | 2.6 | 1.8 | NC |
| | ribonucleoprotein R (Hnrpr) | | | | | |
| BB490701 | Histone 1, H1e (Hist1h2be) | Nucleosome assembly | 1.5 | −2.1 | −1.7 | NC |
| AK009007 | Homeobox only domain (Hod) | Heart development | −2.4 | 2.1 | 1.5 | NC |
| AF208292 | Homeodomain interacting protein | DNA damage | −2.1 | 2.3 | 1.9 | NC |
| | kinase 2 (Hipk2) | response; apoptosis | | | | |
| AI835088 | Homocysteine-inducible, | Stress response | 3.5 | −2.8 | −3.0 | NC |
| | endoplasmic reticulum stress- | | | | | |
| | inducible, ubiquitin-like domain | | | | | |
| | member 1 (Herpud1) | | | | | |
| NM_022331 | Homocysteine-inducible, | Stress response | 3.6 | −2.7 | −3.0 | NC |
| | endoplasmic reticulum stress- | | | | | |
| | inducible, ubiquitin-like domain | | | | | |
| | member 1 (Herpud1) | | | | | |
| AK021220 | Hydroxyacylglutathione hydrolase- | Pyruvate metabolism | −2.1 | 2.5 | 1.8 | NC |
| | like (Haghl) | | | | | |
| AK012748 | Hydroxyacylglutathione hydrolase- | Pyruvate metabolism | −2.0 | 2.1 | 2.0 | NC |
| | like (Haghl) | | | | | |
| AV274826 | IBR domain containing 2 (Ibrdc2) | Ubiquitin cycle | 1.9 | −2.8 | −2.1 | NC |
| BB222675 | Inner membrane protein, | Control of | −3.6 | 7.6 | 4.9 | NC |
| | mitochondrial (Immt) | mitochondrial cristae | | | | |
| | | morphology | | | | |
| BB434111 | Inositol 1,4,5-trisphosphate 3- | Signal transduction | −3.4 | 2.7 | 2.5 | NC |
| | kinase B (Itpkb) | | | | | |
| BB345784 | Insulin receptor substrate 1 (Irs1) | Insulin receptor | 2.0 | −3 | −1.7 | NC |
| | | signaling pathway | | | | |
| BG075165 | Insulin-like growth factor 1 (Igf1) | Anti-apoptosis; organ | −1.9 | 2 | 2.3 | NC |
| | | biogenesis | | | | |
| BC003209 | Integrator complex subunit 3 (Ints3) | snRNA processing | 1.8 | −2 | −2.2 | NC |
| BC008626 | Intercellular adhesion molecule | Defense response; cell | 3.4 | −3.1 | −2.7 | NC |
| | (Icam1) | adhesion | | | | |
| AI481797 | Interferon activated gene 205 | regulation of cell | 4.3 | −3.8 | −2.2 | NC |
| | (Ifi205) | proliferation | | | | |
| BB193024 | Interferon induced transmembrane | Unknown | 2.3 | −3.4 | −3.3 | NC |
| | protein 6 (Ifitm6) | | | | | |
| NM_013562 | Interferon-related developmental | Muscle cell | 2.2 | −2.1 | −1.6 | NC |
| | regulator 1 (Ifrd1) | differentiation | | | | |
| BC016576 | Isochorismatase domain containing | Metabolism | −1.8 | 2.4 | 2.8 | NC |
| | 1 (Isoc1) | | | | | |
| NM_008416 | Jun-B oogene (Junb) | AP-1 transcription | 1.7 | −2.2 | −2.0 | NC |
| | | factor subunit; | | | | |
| | | transcription regulation | | | | |
| NM_021566 | Jutophilin 2 (Jph2) | Eelevation of cytosolic | −1.7 | 2.5 | 2.1 | NC |
| | | calcium ion | | | | |
| | | coentration; | | | | |
| | | development | | | | |
| BB328076 | Kelch-like 24 (Khl24) | Ion transport | 2.6 | −2.7 | −2.2 | NC |
| AK018314 | Kelch-like 24 (Khl24) | Ion transport | 1.6 | −2.1 | −2.1 | NC |
| BB126077 | Kyphoscoliosis peptidase (Ky) | Muscle development | −3.1 | 2.2 | 1.8 | NC |
| L20048 | L20048 | Immune response | 2.1 | −3.5 | −1.9 | NC |
| NM_029796 | Leucine-rich alpha-2-glycoprotein | Cell growth and | 3.3 | −3.5 | −2.9 | NC |
| | (Lrg-pending) | differentiation | | | | |
| BC019794 | Leucine-rich repeat-containing 3b | Protein biosynthesis | −2.1 | 2.4 | 1.9 | NC |
| | (Lrrc3b) | | | | | |
| AK015134 | Leucine-rich repeat-containing 52 | Protein biosynthesis | 5.7 | −2.7 | −2.6 | NC |
| | (Lrrc52) | | | | | |
| BB333759 | Leucine-rich repeat-containing 8c | Protein biosynthesis | 2.0 | −2.1 | −1.7 | NC |
| | (Lrrc8c) | | | | | |
| D17444 | Leukemia inhibitory factor receptor | Positive regulation of | 1.8 | −2.4 | −2.0 | NC |
| | (Lifr) | cell proliferation | | | | |
| BC004826 | Lutheran blood group (Auberger b | Cell adhesion | −1.5 | 2.1 | 1.6 | NC |
| | antigen iluded) (Bcam) | | | | | |
| NM_010741 | Lymphocyte antigen 6 complex, | Defense response | 2.6 | −2.2 | −2.1 | NC |
| | locus C (Ly6c) | | | | | |
| BM241485 | Macrophage activation 2 (Mpa21) | Immune response | 5.3 | −4.2 | −3.6 | NC |
| BB257769 | MAD homolog 1 (Smad1) | Transcription | 1.8 | −2.4 | −1.9 | NC |
| | | regualtion; MAPKKK | | | | |
| | | cascade | | | | |
| NM_010809 | Matrix metalloproteinase 3 (Mmp3) | Extracellular matrix | 7.4 | −3.6 | −2.9 | NC |
| | | remodeling | | | | |
| NM_027209 | Membrane-spanning 4-domains, | Signal transduction | 3.1 | −2.3 | −1.7 | NC |
| | subfamily A, member 6B (Ms4a6b) | | | | | |
| NM_026835 | Membrane-spanning 4-domains, | Signal transduction | 7.1 | −3.3 | −2.2 | NC |
| | subfamily A, member 6d (Ms4a6d) | | | | | |
| NM_013602 | Metallothionein 1 (Mt1) | NO-mediated signal | 4.5 | 3 | −2.4 | NC |
| | | transduction | | | | |
| AA796766 | Metallothionein 2 (Mt2) | Oxidative stress | 15.6 | −6.8 | −5.5 | NC |
| | | response | | | | |
| NM_013594 | Methyl-CpG binding domain | Transcription | 3.1 | −3.6 | −2.6 | NC |
| | protein 1 (Mbd1) | regulation; DNA | | | | |
| | | methylation | | | | |
| AK007371 | Methyl-CpG binding domain | DNA methylation | 2.4 | −3.3 | −2.5 | NC |
| | protein 1 (Mbd1) | | | | | |
| AK007371 | Methyl-CpG binding domain | DNA methylation | 2.1 | −2.1 | −2.0 | NC |
| | protein 1 (Mbd1) | | | | | |
| BF121558 | Methyl-CpG binding domain | DNA methylation | 2.1 | −2.5 | −1.8 | NC |
| | protein 1 (Mbd1) | | | | | |
| BI155184 | Methylcrotonoyl-Coenzyme A | Metabolism | −2.1 | 2.2 | 1.7 | NC |
| | carboxylase 2 (beta) (Mccc2) | | | | | |
| BG074706 | Microtubule-actin crosslinking | Mesoderm formation; | −2.2 | 3.4 | 2.7 | NC |
| | factor 1 (Macf1) | cell motility; cell cycle | | | | |
| | | arrest | | | | |
| C79823 | Mitochondrial ribosomal protein | Intracellular protein | −2.2 | 2.1 | 2.4 | NC |
| | L45 (Mrpl45) | transport | | | | |
| NM_016693 | Mitogen-activated protein kinase | MAPK signaling | 5.9 | −4.1 | −3.2 | NC |
| | kinase kinase 6 (Map3k6) | | | | | |
| BC026425 | Motile sperm domain containing 2 | Cell motility | 2.1 | −2.8 | −1.7 | NC |
| | (Mospd) | | | | | |
| M30697 | Multidrug resistae protein (MDR) | Drug transport | 2.5 | −3.3 | −2.6 | NC |
| | (Acb1a) | | | | | |
| BI076714 | mVL30-1 retroelement | Unknown | 3.5 | −3.1 | −2.1 | NC |
| AI326984 | Myosin binding protein C, fast-type | Muscle contraction; | 1.7 | −2.5 | −3.2 | NC |
| | (Mybpc2) | cell adhesion | | | | |
| AW546141 | Myristoylated alanine rich protein | Cytoskeleton | −2.0 | 2.3 | 1.7 | NC |
| | kinase C substrate (Marcks) | organization | | | | |
| BG070037 | Neuronal PAS domain protein 2 | Two-component signal | 1.7 | −2.7 | −2.6 | NC |
| | (Npas2) | transduction system | | | | |
| | | (phosphorelay) | | | | |
| NM_008808 | NM_008808 | Cell cycle regulation; | −2.0 | 2.3 | 1.7 | NC |
| | | angiogenesis | | | | |
| NM_010929 | Notch gene homolog 4, | Patterning of blood | 2.0 | −2.4 | −2.2 | NC |
| | (Drosophila) (Notch4) | vessels; cell fate | | | | |
| | | determination | | | | |
| AY061760 | Nuclear factor, interleukin 3, | Transcription | 2.5 | −3.1 | −2.5 | NC |
| | regulated (Nfil3) | regulation | | | | |
| BB811478 | Nucleoplasmin 3 (Npm3) | rRNA processing | −2.1 | 2.2 | 2.1 | NC |
| BB534069 | OTU domain, ubiquitin aldehyde | Ubiquitin cycle; | −2.3 | 2.1 | 2.0 | NC |
| | binding 1 (Otub1) | immune response | | | | |
| X63440 | P19-protein tyrosine phosphatase | Cell adhesion; immune | 2.2 | −2.2 | −1.5 | NC |
| | (Ptpn12) | response | | | | |
| BG076140 | p53 regulated PA26 nuclear protein | Cell cycle arrest | 2.1 | −2.8 | −2.9 | NC |
| | (Sestrin 1) (Sesn1) | | | | | |
| BM237933 | p53 regulated PA26 nuclear protein | Cell cycle arrest | 1.5 | −2.2 | −2.4 | NC |
| | (Sestrin 1) (Sesn1) | | | | | |
| AV016566 | p53 regulated PA26 nuclear protein | Cell cycle arrest | 2.3 | −3.1 | −2.3 | NC |
| | (Sestrin 1) (Sesn1) | | | | | |
| BG076140 | p53 regulated PA26 nuclear protein | Cell cycle arrest | 2.9 | −4 | −3.5 | NC |
| | (Sestrin1, Sesn1) | | | | | |
| BM121149 | Pellino 2(peli2) | Modulation of IL-1 | 1.8 | −2.1 | −1.9 | NC |
| | | and TPS signaling | | | | |
| AK004331 | Peptidylprolyl isomerase | Calcium signaling | −2.1 | 2.3 | 1.8 | NC |
| | (cyclophilin)-like 1 (Ppil1) | | | | | |
| BB757992 | Period homolog 3 (Per3) | Circadian rhythm | −3.4 | 2.6 | 2.3 | NC |
| NM_134025 | Peroxisomal biogenesis factor 12 | Protein transport | 2.5 | −2 | −2.4 | NC |
| | (Pex12) | | | | | |
| BI663145 | PHD finger protein 15 (Phf15) | Unknown | 2.4 | −2.1 | −2.1 | NC |
| NM_138755 | PHD finger protein 21A (Phf21a) | Transcription | −1.7 | 2 | 2.1 | NC |
| | | regulation | | | | |
| BC011470 | Phosphatidylinositol binding | Receptor mediated | 2.1 | −2.1 | −1.5 | NC |
| | clathrin assembly protein (Picalm) | endocytosis | | | | |
| NM_019798 | Phosphodiesterase 4A, cAMP | Inactivation of cAMP | −2.0 | 2.1 | 1.8 | NC |
| | specific (Pde4a) | and cGMP | | | | |
| AU015378 | Phosphodiesterase 7A (Pde7a) | Signal transduction | 3.1 | −2.8 | −2.2 | NC |
| AK005158 | Phospholipase A2 group VII | Inflammation; lipid | 3.7 | −2.3 | −2.2 | NC |
| | (platelet-activating factor | catabolism | | | | |
| | acetylhydrolase, plasma) (Pla2g7) | | | | | |
| BM228590 | Phospholipase D1 (Pld1) | Glycerophospholipid | 25 | −3 | −2.0 | NC |
| | | metabolism; | | | | |
| | | intracellular signaling | | | | |
| | | cascade | | | | |
| BM228590 | Phospholipase D1 (Pld1) | Glycerophospholipid | 2.0 | −2 | −1.6 | NC |
| | | metabolism; | | | | |
| | | intracellular signaling | | | | |
| | | cascade | | | | |
| BG073502 | Pleckstrin homology domain | Regulation of Rho | 1.7 | −2.4 | −2.0 | NC |
| | containing, family G (with RhoGef | protein signal | | | | |
| | domain) member 1 (Plekhg1) | transduction | | | | |
| AF065162 | Potassium channel, subfamily K, | Ion transport | −2.5 | 2.5 | 1.7 | NC |
| | member 3 (Kcnk3) | | | | | |
| BF467278 | Potassium channel, subfamily K, | Ion transport | −2.7 | 6.7 | 3.9 | NC |
| | member 3 (Kcnk3) | | | | | |
| NM_008419 | Potassium voltage-gated channel, | Ion transport | 2.6 | −3.2 | −2.4 | NC |
| | shaker-related subfamily, member 5 | | | | | |
| | (Kcna5) | | | | | |
| NM_008880 | Pphospholipid scramblase 2 | Myeloid cell | 1.8 | −2 | −1.8 | NC |
| | (Plscr2) | differentiation | | | | |
| | Procollagen-proline, 2-oxoglutarate | | | | | |
| BB253720 | 4-dioxygenase (proline 4- | Protein metabolism | −2.0 | 2.4 | 1.9 | NC |
| | hydroxylase), alpha 1 polypeptide | | | | | |
| | (P4ha1) | | | | | |
| BM243379 | Prohibitin (Phb) | DNA replication; mast | 1.6 | −2.2 | −2.1 | NC |
| | | cell activation | | | | |
| NM_011172 | Proline dehydrogenase (Prodh) | Glutamate biosynthesis | 1.9 | −2.2 | −2.0 | NC |
| AB006361 | Prostaglandin D synthetase (Ptgds) | Prostaglandin | 3.1 | −2.6 | −2.0 | NC |
| | | biosynthesis | | | | |
| AK020120 | Protein arginine N- | Embryonic | −2.2 | 2.1 | 1.8 | NC |
| | methyltransferase 1 (Prmt1) | development | | | | |
| BF179910 | Protein tyrosine phosphatase 4a1 | Positive regulation of | 1.8 | −2 | −1.9 | NC |
| | (Ptp4a1) | cell migration; | | | | |
| | | development | | | | |
| AI503166 | Protein tyrosine phosphatase, | DNA integration | 1.7 | −2.2 | −2.0 | NC |
| | receptor-type, F interacting protein, | | | | | |
| | binding protein 2 (Ppfibp2) | | | | | |
| BC019123 | RAD52 homolog (S. cerevisiae) | DNA repair | 1.8 | −2.1 | −1.8 | NC |
| | (Rad52) | | | | | |
| BB106402 | RAN binding protein 9 (Ranbp9) | Signal transduction | 3.7 | −3.4 | −2.6 | NC |
| AV291679 | Ras association (RalGDS/AF-6) | Negative regulation of | 3.3 | −2.7 | −2.2 | NC |
| | domain family 4 (Neuropeptide | progression through | | | | |
| | signaling) | cell cycle | | | | |
| BC018275 | Ras homolog gene family, member | Angiogenesis; | 1.7 | −2.2 | −2.1 | NC |
| | B (RhoB) | apoptosis | | | | |
| NM_133955 | Ras homolog gene family, member | G1/S transition of | 3.4 | −2.8 | −2.5 | NC |
| | U (Arhu) (Rhou) | mitotic cell cycle; actin | | | | |
| | | cytoskeleton | | | | |
| | | organization and | | | | |
| | | biogenesis; regulation | | | | |
| | | of cell shape | | | | |
| BB217136 | RAS, dexamethasone-induced 1 | Cell growth | 2.8 | −2.6 | −3.3 | NC |
| | (Rasd1) | suppression | | | | |
| BB003229 | RasGEF domain family, member | Cell division | 3.1 | −2.2 | −2.4 | NC |
| | 1B (Rasgef1b) | | | | | |
| NM_019662 | Ras-related associated with diabetes | Small GTPase | −2.8 | 2.5 | 2.4 | NC |
| | (Rrad) | mediated signal | | | | |
| | | transduction | | | | |
| BM194994 | REST corepressor 1 (Rcor1) | Transcription | −1.5 | 2.6 | 3.3 | NC |
| | | regulation; chromatin | | | | |
| | | modification | | | | |
| BG916957 | Restin-like 2 (Rsnl2) | Unknown | −1.8 | 2 | 1.9 | NC |
| BF011461 | Retinoblastoma binding protein 4 | DNA damage response | −2.1 | 2.9 | 2.1 | NC |
| | (Rbbp4) | | | | | |
| NM_023462 | Retinol binding protein 7, cellular | Transport | 1.8 | −2.3 | −1.6 | NC |
| | (Rbp7) | | | | | |
| BC025502 | Rho GTPase activating protein 24 | GTPase activation; | 2.2 | −2 | −1.8 | NC |
| | (Arhgap24) | signaling | | | | |
| BB493265 | RNA, U22 small nucleolar | Protein binding | 2.2 | −2.5 | −1.8 | NC |
| NM_013650 | S100 calcium binding protein A8 | Cell proliferation; | 7.1 | −14.3 | −19.9 | NC |
| | (calgranulin A) (S100a8) | calcium signaling | | | | |
| NM_009114 | S100 calcium binding protein A9 | Cell proliferation; | 7.1 | −15.9 | −14.0 | NC |
| | (calgranulin B) (S100a9) | calcium signaling | | | | |
| NM_054037 | Secretoglobin, family 3A, member | Cytokine activity | 2.6 | −3.3 | −2.2 | NC |
| | 1 (Scgb3a1) | | | | | |
| BQ176610 | Sema domain, seven | Patterning of blood | 4.3 | −4.9 | −3.9 | NC |
| | thrombospondin repeats (type 1 and | vessels; brahing | | | | |
| | type 1-like), transmembrane domain | morphogenesis | | | | |
| | (TM) and short cytoplasmic | | | | | |
| | domain, (semaphorin) 5A (Sema5a) | | | | | |
| BM244064 | Serine iorporator 3 (Seri3) | Induction of apoptosis | 2.6 | −2.1 | −1.8 | NC |
| BB794710 | Serine palmitoyltransferase, long | Metabolism | 2.2 | −2.2 | −2.1 | NC |
| | chain base subunit 2 (Sptlc2) | | | | | |
| BQ174721 | SERTA domain containing 4 | Growth inhibition | −1.6 | 2.3 | 2.0 | NC |
| | (Sertad4) | | | | | |
| BG069700 | SET domain containing (lysine | Chromatin | −2.3 | 2.2 | 1.9 | NC |
| | methyltransferase) 8)Setd8) | modification | | | | |
| BM229104 | SET translocation (Set) | Nucleosome assembly | −1.6 | 2 | 2.0 | NC |
| BF134272 | SET translocation (Set) | Nucleosome assembly | −2.0 | 2.5 | 2.0 | NC |
| BC027262 | Similar to metallothionein 1 (Mt1) | Nitric oxide mediated | 2.7 | −2.9 | −2.7 | NC |
| | | signal transduction | | | | |
| BC011158 | Similar to serine protease inhibitor- | Protease inhibition | 2.7 | −2.5 | −2.4 | NC |
| | 2 related sequee 1 (Serpina3m) | | | | | |
| NM_011338 | Small inducible cytokine A9 | Chemotaxis; immune | 2.6 | −2.5 | −2.1 | NC |
| | (Scya9) (Ccl9) (Mip-1□) | response | | | | |
| AF128196 | Small inducible cytokine A9 | Chemotaxis; immune | 2.4 | −2.4 | −2.0 | NC |
| | (Scya9) (Ccl9) (Mip-1□) | response | | | | |
| NM_018866 | Small inducible cytokine subfamily | Inflammation | 6.3 | −5.4 | −4.4 | NC |
| | B (Cys-X-Cys), member 13 | | | | | |
| | (Scyb13) (Cxcl13) | | | | | |
| BF578669 | Smoothelin (Smtn) | Actin anchor | −2.0 | 2 | 1.5 | NC |
| AV244484 | Solute carrier family 10 | Transport | 7.7 | −7.2 | −5.3 | NC |
| | (sodium/bile acid cotransporter | | | | | |
| | family), member 6 (Slc10a6) | | | | | |
| BC003438 | Solute carrier family 39 (iron- | Ion transport | 2.2 | −2.3 | −2.1 | NC |
| | regulated transporter), member 1 | | | | | |
| | (Slc40a1) | | | | | |
| NM_021398 | Solute carrier family 43, member 3 | Transport | 2.9 | −2.4 | −2.0 | NC |
| | (Slc43a3) | | | | | |
| BC024519 | Solute carrier family 45, member 3 | Transport | 2.4 | −2.4 | −1.8 | NC |
| | (Slc45a3) | | | | | |
| AK016616 | Sphingosine kinase 2 (Sphk2) | Blood vessel | −2.8 | 2.2 | 1.5 | NC |
| | | development; anti- | | | | |
| | | apoptosis; cell | | | | |
| | | proliferation | | | | |
| AK004781 | SRY-box containing gene 17 | Transcription | 4.1 | −2.7 | −1.6 | NC |
| | (Sox17) | regulation | | | | |
| AK002700 | Sulfotransferase family 1A, phenol- | Steroid metabolism | 4.0 | −3.7 | −3.7 | NC |
| | preferring, member 1 (Sult1a1) | | | | | |
| AV296217 | Syntaxin 3 (Stx3) | Intracellular protein | 2.4 | −2.1 | −1.6 | NC |
| | | transport | | | | |
| NM_023719 | Thioredoxin interacting protein | Response to oxidative | 1.8 | −3.6 | −3.5 | NC |
| | (Txnip) | stress | | | | |
| NM_007434 | Thymoma viral proto-oogene 2 | Regulation of JNK | −1.8 | 2.7 | 1.7 | NC |
| | (Akt2) | cascade; cell cycle | | | | |
| | | regulation | | | | |
| BI788452 | Tissue inhibitor of | Inactivation of | 3.2 | −3.3 | −3.1 | NC |
| | metalloproteinase 4 (Timp4) | metalloproteinases | | | | |
| BB328405 | Tissue inhibitor of | Inactivation of | 5.6 | −5 | −2.8 | NC |
| | metalloproteinase 4 (Timp4) | metalloproteinases | | | | |
| NM_021484 | Titin immunoglobulin domain | Muscle development | 4.8 | −3.4 | −3.0 | NC |
| | protein (Myotilin, Myot) | | | | | |
| NM_021297 | Toll-like receptor 4 (TLR4) | Inflammation; I- | 3.2 | −3.7 | −2.6 | NC |
| | | kappaB kinase/NF- | | | | |
| | | kappaB cascade; one- | | | | |
| | | half of LPS receptor | | | | |
| | | (with CD14) | | | | |
| AF185285 | Toll-like receptor 4 (TLR4) | Inflammation; I- | 2.2 | −2.4 | −1.9 | NC |
| | | kappaB kinase/NF- | | | | |
| | | kappaB cascade; one- | | | | |
| | | half of LPS receptor | | | | |
| | | (with CD14) | | | | |
| NM_053085 | Transcription factor 23 (Tcf23) | Transcription | 3.5 | −3.6 | −2.3 | NC |
| | | regulation | | | | |
| BB405795 | Transcription factor Dp 2 (Tfdp2) | Regulation of | 2.8 | −2.5 | −2.2 | NC |
| | | progression through | | | | |
| | | cell cycle | | | | |
| AF384055 | Transcription factor myocardin | Regulation of cell | −1.6 | 2.8 | 4.0 | NC |
| | (Myocd) | growth by extracellular | | | | |
| | | stimulus; | | | | |
| | | vasculogenesis | | | | |
| NM_021897 | Transformation related protein 53 | Stress response; | 4.8 | −5 | −3.5 | NC |
| | inducible nuclear protein 1 | apoptosis | | | | |
| | (Trp53inp1) | | | | | |
| BG793483 | Transforming growth factor, beta | Regulation of cell | 2.4 | −2.4 | −2.3 | NC |
| | receptor II (Tgfbr2) | proliferation | | | | |
| AK019530 | Transforming, acidic coiled-coil | Cell division | −2.1 | 3 | 3.2 | NC |
| | containing protein (Tacc1) | | | | | |
| BI466416 | Transforming, acidic coiled-coil | Centrosome/mitotic | 2.6 | −4.1 | −2.5 | NC |
| | containing protein 2 (Tacc2) | spindle dynamics and | | | | |
| | | gene regulation | | | | |
| BC004057 | Transforming, acidic coiled-coil | Centrosome/mitotic | 2.8 | −2.5 | −2.1 | NC |
| | containing protein 2 (Tacc2) | spindle dynamics and | | | | |
| | | gene regulation | | | | |
| BB550124 | Transglutaminase 2, C polypeptide | G-protein signaling, | 2.0 | −2.5 | −1.7 | NC |
| | (Tgm2) | coupled to IP3 second | | | | |
| | | messenger | | | | |
| | | (phospholipase C | | | | |
| | | activating) | | | | |
| BB041811 | Transglutaminase 2, C polypeptide | G-protein signaling, | 2.0 | −2.6 | −1.7 | NC |
| | (Tgm2) | coupled to IP3 second | | | | |
| | | messenger | | | | |
| | | (phospholipase C | | | | |
| | | activating) | | | | |
| AW985925 | Transmembrane protein 23 | Regulation of cell | 2.8 | −2.6 | −2.1 | NC |
| | (Tmem23) | proliferation and | | | | |
| | | apoptosis | | | | |
| C77858 | Transmembrane protein 38B | Nucleosome assembly; | 2.1 | −2 | −1.5 | NC |
| | (Tmem38b) | chromosome | | | | |
| | | organization and | | | | |
| | | biogenesis | | | | |
| AV152953 | Transthyretin (Ttr) | Hormone signaling | 4.4 | −2.8 | −2.8 | NC |
| BB354684 | Tribbles homolog 2 (Trib2) | Regulation of MAPK | −1.9 | 2.2 | 1.6 | NC |
| | | activity | | | | |
| BM945528 | Tripartite motif protein 24 (Trim24) | Transcription | 1.6 | −2 | −1.8 | NC |
| | | regulation | | | | |
| D63902 | Tripartite motif protein 25 (Trim25) | Transcription | 1.7 | −2 | −2.1 | NC |
| | | regulation | | | | |
| AF201289 | TSC22-related inducible leucine | Anti-apoptosis; | 2.7 | −2.3 | −2.3 | NC |
| | zipper 3c (Tilz3c) (Dsip1) | transcription regulation | | | | |
| BC008117 | Tubulin alpha (Tuba2) | Microtubule-based | −2.7 | 3.2 | 4.1 | NC |
| | | movement | | | | |
| NM_009446 | Tubulin, alpha 3 (Tuba3) | Microtubule-based | −1.9 | 2.1 | 1.9 | NC |
| | | movement | | | | |
| NM_009447 | Tubulin, alpha 4 (Tuba4) | Microtubule-based | −4.3 | 4.2 | 4.7 | NC |
| | | movement | | | | |
| NM_009447 | Tubulin, alpha 4 (Tuba4) | Microtubule-based | −6.2 | 5.8 | 5.8 | NC |
| | | movement | | | | |
| NM_017379 | Tubulin, alpha 8 (Tuba8) | Microtubule | −3.3 | 2.6 | 3.1 | NC |
| | | cytoskeleton | | | | |
| | | organization and | | | | |
| | | biogenesis | | | | |
| BC005547 | Tubulin, beta 2c (Tubb2c) | Microtubule-based | −1.7 | 2.8 | 2.5 | NC |
| | | movement | | | | |
| BC005738 | Tubulointerstitial nephritis antigen- | Proteolysis; transport | 2.1 | −2 | −1.8 | NC |
| | like (Tinagl) | | | | | |
| NM_007987 | Tumor necrosis factor receptor | Apoptosis | 2.8 | −2.4 | −2.4 | NC |
| | superfamily, member 6 (Tnfrsf6) | | | | | |
| | (Fas) | | | | | |
| BB122084 | Tumorsuppressor St7-like (St7l) | Unknown | 2.3 | −2.2 | −2.1 | NC |
| AV290688 | UDP-N-acetyl-alpha-D- | Protein modification | 3.3 | −2.9 | −2.8 | NC |
| | galactosamine:polypeptide N- | | | | | |
| | acetylgalactosaminyltransferase- | | | | | |
| | like 2 (Galntl2) | | | | | |
| BB667216 | Von Willebrand factor homolog | Cell adhesion; blood | 2.3 | −2.1 | −1.7 | NC |
| | (Vwf) | coagulation | | | | |
| AV286265 | Xanthine dehydrogenase (Xdh) | Metabolism | 3.9 | −3.6 | −3.0 | NC |
| BB326368 | Zi finger and BTB domain | Negative regulation of | 5.1 | −3.6 | −1.8 | NC |
| | containing 16 (Zbtb16) | cell proliferation; | | | | |
| | | skeletal development | | | | |
| BM115255 | Zi finger and BTB domain | Negative regulation of | 7.5 | −4 | −1.6 | NC |
| | containing 16 (Zbtb16) | cell proliferation; | | | | |
| | | skeletal development | | | | |

Genes Altered by CH and DOX + CBZ Ttreatinent, But Not DOX or CBZ Alone

| | | | | | | |
|---|---|---|---|---|---|---|
| BB329527 | Activating signal cointegrator 1 | ATP-dependent | 2.0 | −2.1 | NC | NC |
| | complex subunit 3 (Ascc3) | helicase activity | | | | |
| AJ311773 | ART3 mon(ADP- | Protein modification | 1.6 | −2.1 | NC | NC |
| | ribosyl)transferase (art3 gene), | | | | | |
| | splice variant 5 | | | | | |
| BE853170 | cDNA | Unknown | 1.7 | −2.1 | NC | NC |
| BC024802 | cDNA | Unknown | −2.2 | 2 | NC | NC |
| AV277339 | cDNA | Unknown | −2.2 | 2 | NC | NC |
| NM_007868 | Dystrophin, muscular dystrophy | Muscle development | 1.8 | −2.3 | NC | NC |
| | (Dmd) | | | | | |
| NM_138953 | ELL-related RNA polymerase II, | Transcription | 2.7 | −2.4 | NC | NC |
| | elongation factor (E112) | | | | | |
| BQ174518 | EST | Unknown | 1.6 | −2.1 | NC | NC |
| BB009122 | FERM domain containing 4B | Cytoskeletal protein | 1.7 | −2.3 | NC | NC |
| | (Frmd4b) | binding | | | | |
| BC024546 | Homeobox only domain (Hod) | Heart development | −2.6 | 2.1 | NC | NC |
| AA183642 | Macrophage scavenger receptor 1 | Receptor mediated | 2.7 | −2.2 | NC | NC |
| | (Msr1) | endocytosis | | | | |
| AA250031 | Metastasis suppressor 1 (Mtss1) | Cell motility; cell | −2.2 | 2.1 | NC | NC |
| | | adhesion; muscle | | | | |
| | | development | | | | |
| BB745947 | Nuclear transport factor 2-like | Protein import into | 2.0 | −2 | NC | NC |
| | export factor 2 (Nxt2) | nucleus | | | | |
| AV133559 | Potassium channel, subfamily T | Ion transport | 2.4 | −2.1 | NC | NC |
| | member 2 (Kcnt2) | | | | | |
| BM248133 | Potassium voltage-gated channel, | Ion transport | −1.8 | 2.1 | NC | NC |
| | subfamily Q, member 1 (Kcnq1) | | | | | |
| BC025837 | SH3-binding kinase 1 (Sbk1) | Signal transduction | 1.7 | −2.1 | NC | NC |
| BB486599 | ST8 alpha-N-acetyl-neuraminide | Carbohydrate | 24 | −3.1 | NC | NC |
| | alpha-2,8-sialyltransferase 6 | biosynthesis | | | | |
| | (St8sia6) | | | | | |
| NM_011430 | Synuclein, gamma (Sg) | Unknown | −1.6 | 2.1 | NC | NC |
| AW540790 | Transmembrane protein 38B | Nucleosome assembly; | 2.3 | −2.1 | NC | NC |
| | (Tmem38b) | chromosome | | | | |
| | | organization and | | | | |
| | | biogenesis | | | | |

Of the 472 “cardiac hypertrophy-specific” genes that were altered in response to treatment with doxycycline and carbamazepine, 453 and 98 were also altered when either doxycycline or carbamazepine alone was used, when statistical parameters were lifted (i.e., average fold-changes irrespective of statistical measures). The remaining 19 genes were only altered in mice given isoproterenol, compared to normal mice, and in mice given the combination drug therapy (in the opposite direction), but not when either drug was administered alone as seen in TABLE 2. Presumably, these genes represented synergistic transcriptional alterations. These genes included those involved in transport processes, cytoskeleton movement and adhesion, and muscle and heart development. Eighteen of the gene alterations that were determined to be differentially expressed between disease conditions were verified by real-time RT-PCR, see TABLE 3.

TABLE 3

| | | Microarray | | | | Real-time RT-PCR | | | |
|---|---|---|---|---|---|---|---|---|---|
| | | FC | | | | | | | |
| Gene name | Function | CH | Combo | DOX | CBZ | CH | Combo | DOX | CBZ |
| DNA-damage- | Hypoxic stress | 7.4 | −6.2 | −5.6 | — | 13.9 | −2.5 | −5.7 | — |
| inducible transcript 4 | response; cell | | | | | | | | |
| (Ddit4) | growth | | | | | | | | |
| Matrix | Extracellular | 7.4 | −3.6 | −2.9 | — | 5.3 | −1.5 | −3.5 | −2.5 |
| metalloproteinase 3 | matrix | | | | | | | | |
| (Mmp3) | remodeling | | | | | | | | |
| Metallothionein 1 | NO-mediated | 15.6 | −6.8 | −5.5 | — | 19.7 | −2.8 | −14.9 | — |
| (MT2) | signal | | | | | | | | |
| | transduction | | | | | | | | |
| Tubulin, alpha 4 | Microtubule- | −6.2 | 5.8 | 5.8 | — | −26.0 | 27.9 | 36.8 | — |
| (Tuba4) | based | | | | | | | | |
| | movement | | | | | | | | |
| GATA binding | Transcription | −3.0 | 4.0 | 2.7 | 2.1 | −3.3 | 8.6 | 4.9 | 2.0 |
| protein 4 (Gata4) | regulation; heart | | | | | | | | |
| | development | | | | | | | | |
| Serine protease | Acute-phase | 40.5 | −12.7 | −9.7 | −2.3 | 90.5 | −18.4 | −3.8 | −13.9 |
| inhibitor 2-2 (Spi2-2) | response; | | | | | | | | |
| (Serpin3n) | inflammation | | | | | | | | |
| Transformation | Stress response; | 4.8 | −5.0 | −3.5 | — | 6.1 | Red | −1.9 | −3.0 |
| related protein 53 | apoptosis | | | | | | | | |
| inducible nuclear | | | | | | | | | |
| protein 1 (Trp53inp1) | | | | | | | | | |
| NADPH oxidase 4 | Electron | 4.3 | −3.4 | −3.2 | −1.9 | 22.6 | −2.5 | −4.0 | −3.5 |
| (Nox4) | transport; | | | | | | | | |
| | superoxide | | | | | | | | |
| | release | | | | | | | | |
| Gem GTPase (Gem) | Calcium | 2.7 | −2.9 | −2.8 | — | 8.0 | −1.7 | −1.8 | — |
| | channel | | | | | | | | |
| | blockage | | | | | | | | |
| Oncostatin receptor | Inflammation; | 5.1 | −2.9 | −2.6 | −1.7 | Ind | Red | Red | Red |
| (Osmr) | connective | | | | | | | | |
| | tissue | | | | | | | | |
| | production; | | | | | | | | |
| | extracellular | | | | | | | | |
| | matrix turnover | | | | | | | | |
| Phospholipase A2 | Inflammation; | 3.7 | −2.3 | −2.2 | — | 7.5 | −2.0 | −1.9 | −3.5 |
| group VII (platelet- | lipid catabolism | | | | | | | | |
| activating factor | | | | | | | | | |
| acetylhydrolase, | | | | | | | | | |
| plasma) (Pla2g7) | | | | | | | | | |
| SET and MYND | Heart | −2.9 | 3.2 | 2.7 | 1.7 | −1.7 | 4.9 | 1.6 | 3.3 |
| domain containing 1 | development | | | | | | | | |
| (Smyd1) | | | | | | | | | |
| Lipocalin 2 (Lcn2) | Vascular | 27.7 | −16.6 | −13.3 | −1.6 | 64.0 | −7.0 | −9.9 | — |
| | remodeling; | | | | | | | | |
| | apoptosis | | | | | | | | |
| Cyclin-dependent | Cell cycle arrest | 14.6 | −8.6 | −6.5 | — | 128.0 | −13.0 | −7.0 | −2.5 |
| kinase inhibitor 1A | | | | | | | | | |
| (p21) (Cdkn1a) | | | | | | | | | |
| S100 calcium | Cell | 7.1 | −14.3 | −19.9 | — | 5.7 | −137.2 | −181.0 | −2.1 |
| binding protein A8 | proliferation; | | | | | | | | |
| (calgranulin A) | calcium | | | | | | | | |
| (S100a8) | signaling | | | | | | | | |
| S100 calcium | Cell | 7.1 | −15.9 | −14.0 | — | 17.2 | −52.0 | −104.0 | — |
| binding protein A9 | proliferation; | | | | | | | | |
| (calgranulin B) | calcium | | | | | | | | |
| (S100a9) | signaling | | | | | | | | |
| Cyclin G2 (Ccng2) | Cell cycle | 3.4 | −3.8 | −3.1 | — | 9.2 | −2.6 | −5.7 | — |
| | regulation | | | | | | | | |
| Cytokine inducible | Regulation of | 8.2 | −7.9 | −4.9 | −2.4 | 9.9 | −4.0 | −4.0 | −4.3 |
| SH2-containing | cell growth; | | | | | | | | |
| protein 3 (Socs3) | negative | | | | | | | | |
| | regulation of | | | | | | | | |
| | insulin | | | | | | | | |
| | signaling | | | | | | | | |

where N represents normal mice, CH represents isoproterenol-treated mice, DOX represents mice treated with isoproterenol and doxycycline, CBZ represents mice treated with isoproterenol and carbamazepine, and Combo represents mice treated with isoproterenol and doxycycline and carbamazepine. FC represents fold-change. Ind/Red (Induced/reduced) are used instead of fold-changes where no transcript was detected in one of the two samples being compared.

Doxycycline and carbamazepine alter adrenergic receptor signaling and have been examined using Western blot analysis to examine the phosphorylation status of the transcription factor CREB, which is a potent downstream effector of β-adrenergic signaling. Isoproterenol treatment caused a slight increase in the levels of phosphorylated CREB, which remained elevated after treatment with doxycycline. Almost no phosphorylated CREB was detected, however, when mice with cardiac hypertrophy were treated with carbamazepine or the combination of doxycycline and carbamazepine.

The most likely mechanism of action of doxycycline in the context of cardiac hypertrophy is the inhibition of MMPs, which are known to contribute to the hypertrophic phenotype. There is no reason to believe that doxycycline exerts a negative effect on adrenergic signaling, especially considering the fact that a decrease in heart rate in response to doxycycline treatment was not observe, unless it was administered with carbamazepine. This is consistent with previous work, in which non-selective inhibition of MMPs and knock out of specific MMP genes failed to alter blood pressure or heart rate in mice (23)(24)(25). Carbamazepine on the other hand has been correlated with lower blood pressure and heart rates in epileptic patients (26)(27)(28) and has no cardiovascular toxic effects (29). That carbamazepine counters the positive chronotropic effect induced by isoproterenol via depression of β-adrenergic signaling is in accordance with previous work (19) and that carbamazepine inhibits adenylate cyclase in cardiomyocytes in vivo.

While carbamazepine is clearly beneficial to mice after induction of cardiac hypertrophy, there was very little transcriptional alteration in carbamazepine-treated animals compared to those treated with doxycycline alone or with the drug combination. Carbamazepine may activate and/or inhibit cardiac hypertrophy-specific proteins post-transcriptionally, perhaps those transciptionally altered by doxycycline treatment. Regardless of the mechanism there are several cardiac-related genes that were altered by these two drugs when administered alone and/or in combination. For instance, the gene that encodes cAMP-specific phosphodiesterase 4A (PDE4A), which inactivates cAMP, was decreased in response to ISO treatment and restored in response to drug therapy (see TABLE 2). More interestingly, the α-adrenergic receptor (Adra1b), which has been recently demonstrated to prevent a maladaptive cardiac response, was down-regulated in isoproterenol mice and completely restored to basal levels after treatment with the doxycycline and carbamazepine combination (2.3-fold, as seen in TABLE 2).

Carbamazepine interferes with the AC pathway, resulting in an attenuation of the positive chronotropic effect induced by isoproterenol. This attenuation is not observed with doxycycline and is consistent with its mode of action (i.e., MMP inhibition). Phosphorylation of CREB, which lies downstream of AC, was inhibited by carbamazepine treatment, but not by doxycycline treatment, further supporting a role for AC perturbation in the beneficial effects of carbamazepine treatment.

Carbamazepine has also been shown to inhibit Histone Deacetylase (30), transcriptional modulators of genes involved in the hypertrophic response. Increasing evidence demonstrate that inhibition of HDACs, particularly of class II (preferentially expressed in the heart (31)) but also class I might be an efficient therapeutic strategy ((32)(33)(34)). These inhibitory effects on AC and HDACs were demonstrated to occur within the therapeutic range of carbamazepine (19)(30). Valproic Acid is an anti-epileptic, that like carbamazepine has been shown to inhibit HDAC (35). This inhibition has been suggested to explain the ability of valproic acid to attenuate isoproterenol-, angiotensin II- and aortic banding induced cardiac hypertrophy (32)(33). Therefore, we cannot exclude the HDAC inhibition potential of carbamazepine as a rational explanation of its beneficial effect nor can we exclude the involvement of both pathways in carbamazepine therapeutic effect.

In addition, the present invention includes other compounds that have never been related to or given any indication that they would be useful in treating cardiac hypertrophy, yet show some usefulness in such treatment. These compounds may be used alone or in conjunction with other compounds for treatment.

For example, the present invention includes the use of compounds that affect the action on muscular anabolism to prevent myocyte proliferation and/protein synthesis. As such, the present invention includes a pharmaceutical composition having somatostatin (used to treat giantism, acromegalie) which inhibits the secretion of growth hormones, as acromegalie patients usually have a cardiac hypertrophy that is reversed by use of somatostatin. Masoprocol (used to treat actinic keratoses) blocks the myocyte differentiation as shown in cardiomyocytes and this effect may be specific to skeletal muscles.

Another example includes a pharmaceutical composition that affects the action Acetylcholine metabolism. Acetylcholine has many cardiovascular effects including vasodilatation, slows AV conduction, slows heart rate and decrease heart contraction strength. The present invention includes a pharmaceutical composition having a therapeutic amount of isophlurophate (used to treat accommodative esotropia), which inhibits the enzyme that catabolizes acetylcholine, i.e., acetylcholine esterase; ovide (used to treat multiple sclerosis) and inhibits the enzyme that catabolizes acetylcholine, i.e., acetylcholine esterase; and guanidine hydrochloride (used to treat mystenia which is an acetylcholine agonist.

Another example includes a pharmaceutical composition that affects vitaminic actions, as vitamins are known to be involved in many cardio-vascular processes including rennin-angiotensin system and coagulation. Calderol is commonly used to treat a deficiency in Vitamin D. Vitamin D is a negative regulator of the rennin-angiotensin system (RAS) which is one of the most effective strategy to treat cardiac hypertrophy and anti-hypertension drugs is to prevent the action of the RAS. The present inventors recognized that the genetic ablation of the vitamin D receptors results in cardiac hypertrophy. Tretinoin is commonly used to treat a deficiency in Vitamin A. Vitamin A or all-trans retinoic acid has been shown in vitro to inhibit angiotensin II and its effect leading to cardiac hypertrophy and cardiac remodeling.

Another example includes a pharmaceutical composition that create a peripheral vasodilatation and ease the heart workload and include thorazine is currently used as a sedative and psychotropic to treat hypotension; apomorphine is a hypotensive drug used to treat Parkinson and erectile dysfunction; magnesium sulfate used to treat myorelaxant and known to potentiate verapamil and nifepidine hypotension, and has anti-arrhythmic properties; and baclofen used to treat multiple sclerosis and is known to depress excitable cardiac cells.

Yet another example includes oestrogen, such as estrogens, which are known to decrease the synthesis of angiotensin II receptors. Under certain conditions, they can reduce cardiac hypertrophy, and even prevent cardiac hypertrophy such as stilbetin used to treat Menopause.

Yet another example includes HERG channels inhibitors that tend to hyperpolarize cardiomyocytes, decrease blood pressure and heart rate; however, they can also induce long QT, and arrythmias. Such buprenex used as an analgesic.

It is contemplated that any embodiment discussed in this specification can be implemented with respect to any method, kit, reagent, or composition of the invention, and vice versa. Furthermore, compositions of the invention can be used to achieve methods of the invention. It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Kannel W B, Gordon T, Offutt D. Left ventricular hypertrophy by electrocardiogram.
Prevalence, incidence, and mortality in the Framingham study. Ann Intern Med. 1969; 71(1):89-105.
2. Levy D, Garrison R J, Savage D D, Kannel W B, Castelli W P. Prognostic implications of echocardiographically determined left ventricular mass in the Framingham Heart Study. N Engl J. Med. 1990; 322(22):1561-6.
3. Mathew J, Sleight P, Lonn E, Johnstone D, Pogue J, Yi Q, Bosch J, Sussex B, Probstfield J, Yusuf S. Reduction of cardiovascular risk by regression of electrocardiographic markers of left ventricular hypertrophy by the angiotensin-converting enzyme inhibitor ramipril. Circulation. 2001; 104(14):1615-21.
4. Verdecchia P, Schillaci G, Borgioni C, Ciucci A, Gattobigio R, Zampi I, Reboldi G, Porcellati C. Prognostic significance of serial changes in left ventricular mass in essential hypertension. Circulation. 1998; 97(1):48-54.
5. Chien K R. Stress pathways and heart failure. Cell. 1999; 98(5):555-8.
6. Frey N, Olson E N. Cardiac hypertrophy: the good, the bad, and the ugly. Annu Rev Physiol. 2003; 65( ):45-79.
7. Lips D J, deWindt L J, van Kraaij D J W, Doevendans P A. Molecular determinants of myocardial hypertrophy and failure: alternative pathways for beneficial and maladaptive hypertrophy. Eur Heart J. 2003; 24(10):883-96.
8. Molkentin J D, Dorn II GW2. Cytoplasmic signaling pathways that regulate cardiac hypertrophy. Annu Rev Physiol. 2001; 63( ):391-426.
9. Selvetella G, Hirsch E, Notte A, Tarone G, Lembo G. Adaptive and maladaptive hypertrophic pathways: points of convergence and divergence. Cardiovasc Res. 2004; 63(3):373-80.
10. Cui H, Green R D. Regulation of the cAMP-elevating effects of isoproterenol and forskolin in cardiac myocytes by treatments that cause increases in cAMP. Biochem Biophys Res Commun. 2003; 307(1): 119-26.
11. Okumura S, Takagi G, Kawabe J, Yang G, Lee M, Hong C, Liu J, Vatner D E, Sadoshima J, Vatner S F, Ishikawa Y. Disruption of type 5 adenylyl cyclase gene preserves cardiac function against pressure overload. Proc Natl Acad Sci USA. 2003; 100(17):9986-90.
12. Poole-Wilson P A. The Cardiac Insufficiency Bisoprolol Study II. Lancet. 1999; 353(9161):1360-1.
13. Goldstein S, Kennedy H L, Hall C, Anderson J L, Gheorghiade M, Gottlieb S, Jessup M, Karlsberg R P, Friday G, Haskell L. Metoprolol CR/XL in patients with heart failure: A pilot study examining the tolerability, safety, and effect on left ventricular ejection fraction. Am Heart J. 1999; 138(6 Pt 1):1158-65.
14. Packer M, Bristow M R, Cohn J N, Colucci W S, Fowler M B, Gilbert E M, Shusterman N H. The effect of carvedilol on morbidity and mortality in patients with chronic heart failure. U.S. Carvedilol Heart Failure Study Group. N Engl J. Med. 1996; 334(21):1349-55.
15. Lowes B D, Gilbert E M, Abraham W T, Minobe W A, Larrabee P, Ferguson D, Wolfel E E, Lindenfeld J, Tsvetkova T, Robertson A D, Quaife R A, Bristow M R. Myocardial gene expression in dilated cardiomyopathy treated with beta-blocking agents. N Engl J. Med. 2002; 346(18):1357-65.
16. Suarez J, Gloss B, Belke D D, Hu Y, Scott B, Dieterle T, Kim Y, Valencik M L, McDonald J A, Dillmann W H. Doxycycline inducible expression of SERCA2a improves calcium handling and reverts cardiac dysfunction in pressure overload-induced cardiac hypertrophy. Am J Physiol Heart Circ Physiol. 2004; 287(5):H2164-72.
17. Wren J D, Bekeredjian R, Stewart J A, Shohet R V, Garner H R. Knowledge discovery by automated identification and ranking of implicit relationships. Bioinformatics. 2004; 20(3):389-98.
18. Wren J D, Bekeredjian R, Stewart J A, Shohet R V, Garner H R. Knowledge discovery by automated identification and ranking of implicit relationships. Bioinformatics. 2004; 20(3):389-98.
19. Chen G, Pan B, Hawver D B, Wright C B, Potter W Z, Manji H K. Attenuation of cyclic AMP production by carbamazepine. J. Neurochem. 1996; 67(5):2079-86.
20. Livak K J, Schmittgen T D. Analysis of relative gene expression data using real-time quantitative PCR and the 2(-Delta Delta C(T)) Method. Methods. 2001; 25(4):402-8.
21. Perucca E. Pharmacokinetic interactions with antiepileptic drugs. Clin Pharmacokinet. 1982; 7(1):57-84.
22. Abbruzzese T A, Guzman R J, Martin R L, Yee C, Zarins C K, Dalman R L. Matrix metalloproteinase inhibition limits arterial enlargements in a rodent arteriovenous fistula model. Surgery. 1998; 124(2):328-34; discussion 334-5.
23. Palei A C T, Zaneti R A G, Fortuna G M, Gerlach R F, Tanus-Santos J E. Hemodynamic benefits of matrix metalloproteinase-9 inhibition by doxycycline during experimental acute pulmonary embolism. Angiology. 2005; 56(5):611-7.
24. Hayashidani S, Tsutsui H, Ikeuchi M, Shiomi T, Matsusaka H, Kubota T, Imanaka-Yoshida K, Itoh T, Takeshita A. Targeted deletion of MMP-2 attenuates early LV rupture and late remodeling after experimental myocardial infarction. Am J Physiol Heart Circ Physiol. 2003; 285(3):H1229-35.
25. Isojarvi J I, Ansakorpi H, Suominen K, Tolonen U, Repo M, Myllyla V V. Interictal cardiovascular autonomic responses in patients with epilepsy. Epilepsia. 1998; 39(4):420-6.
26. Persson H, Ericson M, Tomson T. Carbamazepine affects autonomic cardiac control in patients with newly diagnosed epilepsy. Epilepsy Res. 2003; 57(1):69-75.
27. Tomson T, Ericson M, Ihrman C, Lindblad L E. Heart rate variability in patients with epilepsy. Epilepsy Res. 1998; 30(1):77-83.
28. Apfelbaum J D, Caravati E M, Kerns WP2, Bossart P J, Larsen G. Cardiovascular effects of carbamazepine toxicity. Ann Emerg Med. 1995; 25(5):631-5.
29. Beutler A S, Li S, Nicol R, Walsh M J. Carbamazepine is an inhibitor of histone deacetylases. Life Sci. 2005; 76(26):3107-15.
30. McKinsey T A, Zhang C L, Olson E N. Signaling chromatin to make muscle. Curr Opin Cell Biol. 2002; 14(6):763-72.
31. Kee H J, Sohn I S, Nam K I, Park J E, Qian Y R, Yin Z, Ahn Y, Jeong M H, Bang Y, Kim N, Kim J, Kim K K, Epstein J A, Kook H. Inhibition of histone deacetylation blocks cardiac hypertrophy induced by angiotensin II infusion and aortic banding. Circulation. 2006; 113(1):51-9.
32. Kook H, Lepore J J, Gitler A D, Lu M M, Wing-Man Yung W, Mackay J, Zhou R, Ferrari V, Gruber P, Epstein J A. Cardiac hypertrophy and histone deacetylase-dependent transcriptional repression mediated by the atypical homeodomain protein Hop. J Clin Invest. 2003; 112(6):863-71.
33. Backs J, Olson E N. Control of cardiac growth by histone acetylation/deacetylation. Circ Res. 2006; 98(1):15-24.
34. Gottlicher M, Minucci S, Zhu P, Kramer O H, Schimpf A, Giavara S, Sleeman J P, Lo Coco F, Nervi C, Pelicci P G, Heinzel T. Valproic acid defines a novel class of HDAC inhibitors inducing differentiation of transformed cells. EMBO J. 2001; 20(24):6969-78.

What is claimed is:

1. A pharmaceutical composition to ameliorate one or more symptoms of myocardial infarction comprising carbamazepine and doxycycline.

2. The composition of claim 1, wherein the pharmaceutical composition comprises one or more tablets, capsules, gel capsules, liquid syrups, soft gels, aqueous suspensions, edible products or a combination thereof.

3. The composition of claim 2, further comprising one or more colorants, detackifiers, excipients, surfactants, lubricants, stabilizers, coatings, carriers, additives or a combination thereof.

4. The composition of claim 1, further comprising one or more anti-epileptic compounds, matrix metalloproteinase inhibitors, antibiotics, beta-blockers, vasodilators, calcium channel blockers, Angiotensin Converting Enzyme inhibitors, diuretics, alpha-blockers or a combination thereof.

5. A pharmaceutical composition to ameliorate one or more symptoms of myocardial infarction comprising a pharmaceutically effective amount of one or more compounds selected from doxycycline, metastat, MM1270(b), marimastat, BAY 12-9566, batimistat, prinomastat, somatostatin, masoprocol, isophlurophate, ovide, guanidine hydrochloride, calderol, tretinoin, thorazine, apomorphine, magnesium sulfate, stilbetin, buprenex, mixtures and combinations thereof.

6. A pharmaceutical composition to ameliorate one or more symptoms of myocardial infarction comprising an anti-epileptic drug.

7. The composition of claim 6, wherein the anti-epileptic drug comprises carbamazepine.

8. The composition of claim 6, further comprising a matrix metalloproteinase inhibitor.

9. A pharmaceutical composition to ameliorate one or more symptoms of myocardial infarction comprising an anti-epileptic drug and matrix metalloproteinase inhibitor.

10. The composition of claim 9, wherein the anti-epileptic drug comprises carbamazepine and the matrix metalloproteinase inhibitor comprises doxycycline.

11. The composition of claim 9, wherein the anti-epileptic drug and the matrix metalloproteinase inhibitor are administered together in a single pharmaceutical composition.

12. A method of treating patient suffering hypertension, cardiac hypertrophy, myocardial infarction and/or ischemia comprising the steps of:
administering a pharmaceutically effective amount of an anti-epileptic drug and a pharmaceutically effective amount of an matrix metalloproteinase inhibitor to a patient suffering one or more symptoms of hypertension, cardiac hypertrophy, myocardial infarction and/or ischemia.

13. The method of claim 12, wherein the anti-epileptic drug comprises carbamazepine and the matrix metalloproteinase inhibitor comprises doxycycline.

14. The method of claim 12, further comprising the administering one or more anti-Epileptics, matrix metalloproteinases inhibitors, antibiotics, beta-blockers, vasodilators, calcium channel blockers, Angiotensin Converting Enzyme inhibitors, diuretics, alpha-blockers or a combination thereof.

15. A method of treating a patient suffering from myocardial infarction and/or cardiac hypertrophy by modulating the response of one or more cardiac hypertrophy-specific genes comprising the steps of:
administering to the patient thought to be suffering from cardiac hypertrophy a pharmaceutically effective amount of an anti-epileptic drug or a pharmaceutically acceptable salt thereof and a pharmaceutically effective amount of an matrix metalloproteinase inhibitor or a pharmaceutically acceptable salt thereof, wherein one or more cardiac hypertrophy-specific genes are altered in response to treatment with doxycycline and carbamazepine.

16. A method for attenuating one or more complications of hypertension comprising the steps of:
administering a pharmaceutically effective amount of a first compound to affect a β-adrenergic pathway; and
administering a pharmaceutically effective amount of a second compound to affect a α-adrenergic pathway.

17. The method of claim 15, wherein the first compound and the second compound are administered together in a single pharmaceutical composition.

18. The method of claim 15, wherein the first compound comprises carbamazepine or a pharmaceutically acceptable salt thereof and the second compound comprises doxycycline or a pharmaceutically acceptable salt thereof.

19. The method of claim 15, further comprising administering one or more anti-epileptic compounds, matrix metalloproteinase inhibitors, antibiotics, beta-blockers, vasodilators, calcium channel blockers, Angiotensin Converting Enzyme inhibitors, diuretics, alpha-blockers or a combination thereof.

20. method of claim 15, wherein the pharmaceutically effective amount of a first compound and the pharmaceutically effective amount of a second compound comprises one or more tablets, capsules, gel capsules, liquid syrups, soft gels, aqueous suspensions, edible products or a combination thereof.
